(19) United States
(12) Patent Application Publication
(10) Pub. No.: US 2026/0048207 A1
(43) Pub. Date: Feb. 19, 2026

(54) SKIN TREATMENT APPARATUS INCLUDING DRUG METERING AND TRANSPORT DEVICE AND SKIN SUCTION FUNCTION

(71) Applicant: HEALUX CO.,LTD., Seoul (KR)

(72) Inventor: Do Yeun KIM, Seoul (KR)

(73) Assignee: HEALUX CO.,LTD., Seoul (KR)

(21) Appl. No.: 19/367,442

(22) Filed: Oct. 23, 2025

Related U.S. Application Data
(63) Continuation of application No. PCT/KR2024/004624, filed on Apr. 8, 2024.

(30) Foreign Application Priority Data
Apr. 25, 2023 (KR) 10-2023-0053957

(51) Int. Cl. A61M 11/04; A61M 35/00; A61N 7/00; A61N 7/02

(52) U.S. Cl. CPC A61M 11/04; A61M 35/003; A61N 7/02; A61M 2202/0007; A61M 2205/103; A61M 2205/3344; A61M 2205/50; A61M 2205/75; A61M 2210/04; A61N 2007/0008; A61N 2007/0078; A61N 2007/027

(57) ABSTRACT

This invention relates to a medical skin treatment device, comprising a control unit for controlling the operation of the skin treatment device, a power supply unit for supplying power to the medical skin treatment device, and a main body including a drug storage unit for storing medication to be injected into the patient's skin, a transport unit for moving the main body, a handpiece that receives power from the power supply unit of the main body, is controlled by the control unit, and injects the drug into the patient's skin to improve the skin, and a drug supply unit coupled to the drug storage unit that supplies a predetermined amount of drug to the handpiece.

### CROSS-REFERENCE TO RELATED APPLICATIONS

This application is a continuation of International Application No. PCT/KR2024/004624 filed on Apr. 8, 2024, which claims priority to Korean Patent Application No. 10-2023-0053957 filed on Apr. 25, 2023, the entire contents of which are herein incorporated by reference.

### TECHNICAL FIELD

The present disclosure relates to a multi-focus skin treating device that automatically controls transducers and thermoelectric element. The present disclosure relates to a skin treating device including a quantitative drug delivery device and a skin suction feature.

### BACKGROUND ART

Ultrasonic therapy is a method of treating skin using high-frequency waves. This method is primarily used for cosmetic purposes, for cosmetic treatments on areas such as the face and neck. Ultrasonic therapy stimulates collagen fibers in the skin, increasing skin elasticity and preventing the loss of skin elasticity due to aging. Additionally, it promotes metabolic activity in skin cells, stimulating the generation of new skin cells and brightening skin tone. This ultrasonic method is specifically divided into planar ultrasonic and focused ultrasonic methods. Among these, the fat removal effect and safety of the focused ultrasonic method are generally higher than those of the planar ultrasonic method. The working principle of the focused ultrasound method involves placing an ultrasound energy transducer on the skin surface of the targeted fat removal area. It then transmits focused ultrasonic energy into the subcutaneous fat tissue. Through the cavitation phenomenon generated by high-energy ultrasound vibrations, it ruptures the membranes of fat cells in the concentrated area. This allows the contents of the fat cells to flow out and be eliminated through the body's own metabolic processes. This process achieves fat reduction by melting and removing fat tissue in the target area while avoiding damage to surrounding healthy tissue.

Ultrasonic devices generate high temperatures during operation, necessitating the use of thermoelectric elements as cooling mechanisms to dissipate this heat. Conventional methods involve measuring skin temperature using a temperature sensor and then cooling via a thermoelectric element or directly adjusting the ultrasound device. This approach presents issues such as inconvenience for the practitioner and difficulty in achieving effective ultrasound control.

### TECHNICAL PROBLEM

This disclosure relates to a skin treatment device comprising a drug metered delivery device and a skin suction function. The skin treatment device enables simple and effective ultrasound procedures by cooling the skin or automatically controlling the transducer, all without the use of a temperature sensor. Furthermore, the skin treatment device can deliver a metered amount of drug to the skin and suction the skin during treatment to secure it, thereby enabling accurate and effective ultrasound procedures.

However, the technical challenges are not limited to those described above, and other technical challenges may exist.

### TECHNICAL SOLUTION

The medical skin treatment device according to this disclosure comprises a main body including a control unit for controlling the operation of the skin treatment device and a power supply unit for supplying power to the medical skin treatment device, a transport unit for moving the main body, and a handpiece supplied with power from the power supply unit of the main body, controlled by the control unit, and improving the skin by forming lesions based on vibrational energy beneath the patient's skin surface. The handpiece includes a base unit formed on one side of the handpiece and including a concave transducer placement surface, a plurality of ring-shaped transducers formed on the concave transducer placement surface of the base unit, vibrating at a predetermined treatment frequency to transmit vibrational energy to the patient's skin, and a thermoelectric element formed on the concave transducer placement surface of the base unit, formed along the outer periphery of the plurality of transducers, and cooling the plurality of transducers to prevent the patient's skin surface in contact with one side of the handpiece from being burned. The plurality of transducers include a first transducer and a second transducer having a larger diameter than the first transducer. the plurality of transducers have different diameters and form concentric circles, with multiple focal points formed vertically to simultaneously treat different layers beneath the skin.

The depth of the focal points formed beneath the skin by the plurality of transducers of the medical skin treatment device according to this disclosure is proportional to the diameter of each respective the plurality of transducers.

The plurality of transducers of the medical skin treatment device according to the present disclosure radiate vibrational energy simultaneously or sequentially based on user input regarding the treatment area.

The control unit of the medical skin treatment device according to this disclosure acquires the cumulative shot count of the plurality of transducers, acquires the skin temperature corresponding to the cumulative shot count based on at least one of a first table and a first function that correlates the cumulative shot count to skin temperature, and determines the treatment shot count based on the skin temperature corresponding to the cumulative shot count.

The control unit of the medical skin treatment device according to this disclosure acquires a cumulative irradiation time of the plurality of transducers, acquires a skin temperature corresponding to the cumulative irradiation time based on at least one of a second table and a second function that correlates cumulative irradiation time to the skin temperature, and determines a treatment irradiation time based on the skin temperature corresponding to the cumulative irradiation time.

The control unit of the medical skin treatment device according to the present disclosure acquires a cumulative shot count of the plurality of transducers, acquires a cumulative irradiation time of the plurality of transducers, and determines at least one of a treatment shot count and a treatment irradiation time based on at least one of the cumulative shot count and the cumulative irradiation time.

The control unit of the medical skin treatment device according to this disclosure acquires a treatment objective based on user input, automatically provides at least one of an ultrasound treatment shot count and a treatment irradiation time corresponding to the treatment objective, and the treatment objective includes at least one of information regarding a treatment area and a procedure type.

The control unit of the medical skin treatment device according to this disclosure determines the voltage to be applied to the thermoelectric element based on at least one of the skin temperature obtained from at least one of the first table, first function, second table, and second function, and at least one of the third table and third function correlating the skin temperature and the voltage applied to the thermoelectric element. controls the voltage applied to the thermoelectric element to automatically regulate its temperature. At least one of the first table, first function, second table, second function, third table, and third function may differ for each of the transducer frequency, number of formed foci, and treatment objective.

The control unit of the medical skin treatment device according to this disclosure acquires the cumulative shot count of the plurality of transducers, acquires the cumulative irradiation time of the plurality of transducers, determines a voltage to be applied to the thermoelectric element, by applying at least one of the cumulative shot count and the cumulative irradiation time to at least one of a third-third table, a third-third function, a third-fourth table, and a third-fourth function during treatment period, and determines a voltage to be applied to the thermoelectric element, by applying a treatment stoppage time to at least one of a third-fifth table and a third-fifth function during non-treatment period.

The control unit of the medical skin treatment device according to this disclosure acquires a cumulative shot count of the plurality of transducers, acquires a cumulative irradiation time of the plurality of transducers, determines skin temperature, by applying at least one of the cumulative shot count and the cumulative irradiation time to at least one of a first table, a first function, a second table, and a second function during treatment period, and when the skin temperature is equal to or above a predetermined threshold temperature, discharges a predetermined amount of cooling gas, and determines temperature after the cooling gas discharge based on the skin temperature and the predetermined amount of cooling gas.

The medical skin treatment device according to this disclosure comprises a main body including a control unit for controlling the operation of the skin treatment device, a power supply unit for supplying power to the medical skin treatment device, and a drug storage unit for storing a drug to be injected into the patient's skin, a transport unit for moving the main body, a handpiece supplied with power from the power supply unit of the main body, controlled by the control unit, and injecting the drug into the patient's skin to improve the skin, and a drug supply unit coupled to the drug storage unit and supplying a predetermined amount of drug to the handpiece. Wherein the handpiece comprises, a base unit coupled to one side of the handpiece and including a transducer placement surface; a drug injection unit coupled to the drug supply unit, positioned at the center of the base unit, and injecting the drug into the patient's skin, a skin suction unit formed along the outer periphery of the drug injection unit, which sucks in the patient's skin, a filter for filtering drugs and waste materials sucked in by the skin suction unit, and a ring-shaped transducer formed along the outer periphery of the skin suction unit, which vibrates at a predetermined treatment frequency to deliver vibrational energy to the patient's skin.

The drug supply unit of the medical skin treatment device according to this disclosure comprises a pressurized housing having an inlet coupled to the drug storage unit and an outlet coupled to the drug injection unit; a rubber tube located inside the pressurized housing, coupling the inlet and outlet and conveying the drug; a rotating unit located inside the pressurized housing, rotatably coupled to the pressurized housing about a drive shaft and comprising a plurality of rollers, a rotary plate, and a roller support unit; and a drive unit including the drive shaft penetrating the center of the rotating unit, which rotates the rotating unit about the drive shaft; wherein, as the rotating unit rotates, the pressurized housing and the plurality of rollers compress at least a portion of the rubber tube conveying the drug, thereby separating a predetermined amount of drug between the plurality of rollers; and The rotating unit includes a rotary plate with protrusions formed at 120-degree intervals around the drive shaft, a roller support unit coupled to the protrusions of the rotary plate and coupled to the rotation shaft of one of the plurality of rollers, and one of the plurality of rollers coupled to the roller support unit.

The drug supply unit of the medical skin treatment device according to this disclosure, when the medical skin treatment device is deactivated, retracts the plurality of rollers into the interior of the rotary plate so that no pressure is applied to the rubber tube, and when the medical skin treatment device is activated, protrudes the plurality of rollers outward from the rotary plate to apply pressure to the rubber tube.

The drug supply unit of the medical skin treatment device according to this disclosure, when the medical skin treatment device is deactivated, retracts the rotating unit so as not to apply pressure to the rubber tube, and when the medical skin treatment device is activated, move forward the rotating unit to apply pressure to the rubber tube.

According to the present disclosure, when the medical skin treatment device is deactivated, the pressurized housing moving unit included in the pressurized housing moves forward relative to the pressurized housing base unit included in the pressurized housing by the housing drive unit, thereby not applying pressure to the rubber tube. When the medical skin treatment device is activated, the housing drive unit causes the pressurized housing moving unit to retract relative to the pressurized housing base unit, thereby applying pressure to the rubber tube, and the housing drive unit is coupled to the pressurized housing base unit.

The skin suction unit of the medical skin treatment device according to this disclosure sucks in the patient's skin to form negative pressure, includes a pressure sensor for measuring the pressure formed inside the skin suction unit, The control unit controls the drug injection unit to stop injecting drugs when the pressure formed inside the skin suction unit is equal to or greater than a critical pressure, and controls the drug injection unit to inject drugs when the pressure of the skin suction unit is less than the critical pressure.

The control unit of the medical skin treatment device according to this disclosure controls the transducer not to emit vibrational energy when the pressure in the skin suction unit is equal to or greater than the critical pressure, and controls the transducer to emit vibrational energy when the pressure in the skin suction unit is less than the critical pressure.

the medical skin treatment device according to this disclosure changes the depth of focus of the transducer by moving the transducer placement surface up and down based on the depth of focus input by the user.

### ADVANTAGEOUS EFFECTS

The skin treatment device of this disclosure automatically controls the transducer and thermoelectric element for user convenience. The user can perform an ultrasound treatment by selecting the desired procedure. Furthermore, drug is automatically injected, and the patient's skin is suctioned to secure the treatment area, enabling more precise and delicate treatment.

The effects obtainable from the present disclosure are not limited to those mentioned above, and other effects not mentioned will be clearly understood by those skilled in the art from the following description.

### DESCRIPTION OF DRAWINGS

FIG. 1 is a drawing showing a medical skin treatment device according to an embodiment of the present disclosure.

FIG. 2 is a drawing showing a block diagram of various configurations that may be included in the skin treatment device according to an embodiment of the present disclosure.

FIG. 3 is a drawing showing a cross-sectional view of the base unit according to an embodiment of the present disclosure.

FIGS. 4A and 4B are cross-sectional views of the base unit according to an embodiment of the present disclosure.

FIG. 5 is a cross-sectional view of the base unit according to an embodiment of the present disclosure.

FIG. 6 is a drawing for explaining the handpiece according to an embodiment of the present disclosure.

FIG. 7 is a flowchart for explaining the operation of the skin treatment device according to an embodiment of the present disclosure.

FIG. 8 is a flowchart illustrating the operation of the skin treatment device according to an embodiment of the present disclosure.

FIG. 9 is a flowchart illustrating the operation of the skin treatment device according to an embodiment of the present disclosure.

FIG. 10 is a flowchart illustrating the operation of the skin treatment device according to an embodiment of the present disclosure.

FIG. 11 is a diagram showing the drug supply unit according to an embodiment of the present disclosure.

FIGS. 12A and 12B are diagrams showing the operation of the drug supply unit according to an embodiment of the present disclosure.

FIGS. 13A and 13B are diagrams illustrating the operation of the rotating unit according to an embodiment of the present disclosure.

FIGS. 14A, 14B, and 14C are diagrams illustrating the operation of the drug supply unit according to an embodiment of the present disclosure.

FIGS. 15A, 15B, and 15C are diagrams illustrating the operation of the drug supply unit according to an embodiment of the present disclosure.

FIGS. 16A and 16B are diagrams illustrating the base unit according to an embodiment of the present disclosure.

FIGS. 17A and 17B are diagrams illustrating a predetermined function according to an embodiment of the present disclosure.

FIG. 18 is a diagram showing the drug supply unit according to an embodiment of the present disclosure.

### DETAILED DESCRIPTION

Advantages and features of the disclosed embodiments and methods for achieving them will become clear by referencing embodiments which will be described together with the accompanying drawings. However, the present disclosure is not limited to the embodiments to be disclosed below but can be implemented in various different forms, and these embodiments are merely provided to make the disclosure of the present disclosure complete and fully inform those skilled in the art to which the present disclosure pertains in the scope of the present disclosure.

Terms used in the present specification will be briefly described, and disclosed embodiments will be described in detail.

The terms used in the present specification are general terms that are currently widely used as much as possible while considering a function in the present disclosure, but this can vary depending on the intention or cases of a technician who works in the art, the emergence of a new technology, etc. In addition, in specific cases, there are terms arbitrarily selected by the applicant, and in this case, the meaning will be described in detail in the description of the corresponding invention. Accordingly, the terms used in the present disclosure should be defined based on the meanings of the terms and the overall content of the present disclosure rather than simply the names of the terms.

A singular expression used herein includes a plural expression unless the context clearly dictates otherwise. In addition, the plural expression includes the singular expression unless the context clearly dictates otherwise.

Throughout the specification, when a certain portion is described as “including” a certain component, it means further including another component rather than precluding another component unless especially stated otherwise.

In addition, the term “unit” used in the specification means a software or hardware component, and the “unit” performs certain roles. However, the “unit” is not limited to software or hardware. The “unit” may be configured in an addressable storage medium or configured to reproduce one or more processors. Accordingly, as an example, the “unit” includes components such as software components, object-oriented software components, class components, and task components, processes, functions, attributes, procedures, subroutines, segments of program code, drivers, firmware, microcode, circuits, data, database, data structures, tables, arrays, and variables. Functions provided in the components and “units” may be combined with a smaller number of components and “units” or separated into additional components and “units.”

According to one embodiment of the present disclosure, the “unit” may be implemented as a processor and a memory. The term “processor” should be construed broadly to include general-purpose processors, central processing units (CPUs), microprocessors, digital signal processors (DSPs), controllers, microcontrollers, state machines, etc. In some circumstances, the “processor” may also refer to application-specific integrated circuits (ASICs), programmable logic devices (PLDs), field programmable gate arrays (FPGAs), etc. For example, the term “processor” may also refer to a combination of processing devices, such as a combination of a DSP and a microprocessor, a combination of a plurality of microprocessors, a combination of one or more microprocessors coupled with a DSP core, or any other such combination of components.

The term “memory” should be construed broadly to include any electronic component capable of storing electronic information. The term “memory” may refer to various types of processor-readable media, such as a random access memory (RAM), a read-only memory (ROM), a non-volatile RAM (NVRAM), a programmable ROM (PROM), an erasable programmable ROM (EPROM), an electrically erasable PROM (EEPROM), a flash memory, a magnetic or optical data storage, registers, etc. The memory is considered to function in an electronic communication state with the processor when the processor may read information from and/or write information on the memory. The memory integrated in the processor is in electronic communication with the processor.

In the present specification, an actuator means a component capable of providing a driving force. For example, the actuator may include a motor, a linear motor, an electric motor, a DC motor, an AC motor, a linear actuator, an electric actuator, etc., but is not limited thereto.

Hereinafter, the present disclosure will be described in detail with reference to the accompanying drawings so that those skilled in the art to which the present disclosure pertains can easily carry out embodiments. In addition, to clearly describe the present disclosure in the drawings, parts that are not related to the description will be omitted.

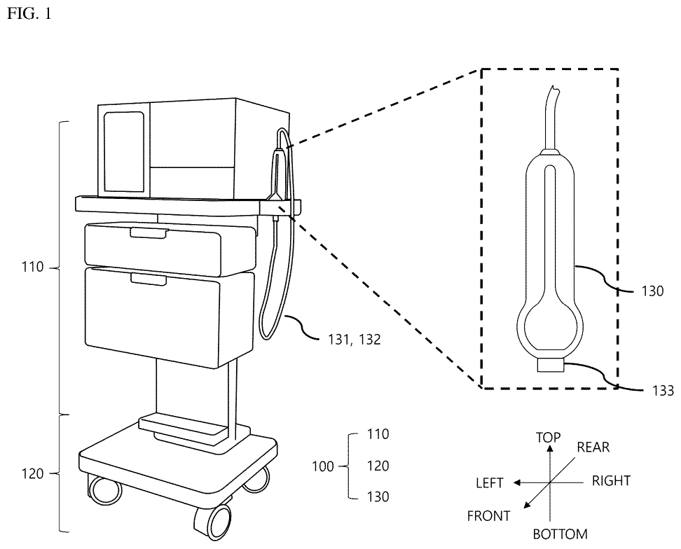

FIG. 1 is a diagram illustrating a medical skin treatment device according to an embodiment of the present disclosure.

The medical skin treatment device 100 may include a main body 110 comprising a control unit 200 and a power supply unit. The main body 110 may further include at least one of a communication unit, a sensor unit, an output unit, and an input unit.

Furthermore, the medical skin treatment device 100 may include a transport unit 120 for moving the main body 110. The transport unit 120 may be located at the bottom of the main body 110. The transport unit 120 may include at least one wheel. The transport unit 120 may include at least one of a caster wheel, an electric wheel, and a Mecanum wheel. The transport unit 120 may include an electric motor, and the electric motor may provide driving force based on user input. Furthermore, the transport unit 120 may be capable of autonomous driving based on control from the control unit 200. However, this is not limited thereto, and the transport unit 120 may be moved by the user's force.

The medical skin treatment device 100 may also include a handpiece 130. The main body 110 may be coupled to the handpiece 130 via a cable 131. The handpiece 130 may be used to treat the patient's skin. The handpiece 130 may include a handle that the user can grip. One side of the handpiece 130 may have a surface formed to contact the patient's skin. The main body 110 can supply power to the handpiece 130 via the cable 131 and transmit or receive control signals via the cable.

The medical skin treatment device 100 may include a drug supply unit 1100 coupled to a drug storage unit and supplying a predetermined amount of drug to the handpiece. The drug supply unit 1100 is formed on the side of the main body 110 and may be detachable. The main body 110 may include a drug storage unit for storing drugs to be injected into the patient's skin. The drug storage unit may include a space for storing drug containers and a cover covering the space. The user can open the cover of the drug storage unit to connect or replace the drug container used for the procedure. a plurality of drug containers containing different drugs can be connected to the drug storage unit. However, this is not limited to this configuration; the drug storage unit may connect to a single drug container containing one drug. The drug supply unit 1100 may be located near the drug storage unit to rapidly supply drugs. The drug storage unit may be coupled to the drug supply unit 1100 via a rubber tube 132. The drug supply unit 1100 may be coupled to the handpiece 130 via a rubber tube 132. The rubber tube 132 may be made of rubber material. However, it is not limited to this; it may be made of a material that is corrosion-resistant to the drug, possesses non-corrosive properties, and exhibits rapid shape compression and recovery. The drug supply unit 1100 will be described in more detail later.

The handpiece 130 can receive power from the power supply unit of the main body 110. The handpiece 130 can be controlled by the control unit 200. The handpiece 130 can improve the skin by having one side contact the patient's skin and forming lesions beneath the patient's skin surface based on vibrational energy. For example, subcutaneous tissue may be destroyed by the vibrational energy, or the tissue temperature may rise due to the vibrational energy, leading to lesion formation. The patient's destroyed tissue may undergo improvement during the recovery process. Furthermore, the destroyed tissue may be adipose tissue; since the handpiece 130 destroys the adipose tissue, it may have the effect of reducing subcutaneous fat in the skin. The components included in the handpiece 130 will be described in detail later. The handpiece 130 may include a base unit 133 formed on one side of the handpiece 130 and including a transducer placement surface 310. The base unit 133 may be formed facing the patient's skin. The components included in the handpiece 130 will be described in detail later.

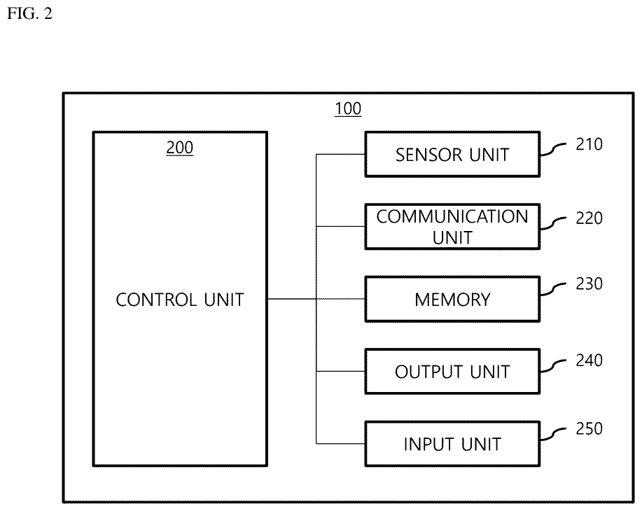

FIG. 2 is a diagram showing a block diagram of various configurations that may be included in a skin treatment device according to an embodiment of the present disclosure.

The skin treatment device 100 may include a sensor unit 210, a communication unit 220, a memory 230, an output unit 240, an input unit 250, and a control unit 200.

The skin treatment device 100 may include a control unit 200. The control unit 200 can control the operation of the skin treatment device 100. For example, the skin treatment device 100 may include a control unit 200 for controlling the operation of at least one of wheels capable of driving the main body 110 and a handpiece. The control unit 200 may include one processor and may include multiple processors. The control unit 200 may be included within the main body 110. If the control unit 200 includes multiple processors, at least some of the multiple processors may be provided at a location physically separated from the main body 110. Furthermore, the skin treatment device 100 may be implemented in various ways, not limited to the above.

According to an embodiment of the present disclosure, the control unit 200 may control the operation of the skin treatment device 100. For example, the skin treatment device 100 may include multiple actuators, and the skin treatment device 100 may control its operation by controlling the operation of the multiple actuators. For example, the control unit 200 can control the operation of the handpiece 130 to perform operations for improving the patient's skin.

The skin treatment device 100 may include a sensor unit 210. The sensor unit 210 can acquire various information using at least one sensor. The sensor unit 210 may be equipped with sensors utilizing measurement means such as pressure, potential, and optics. For example, the sensor unit 210 may include at least one distance measurement sensor or encoder. Additionally, the sensors may include pressure sensors, infrared sensors, LED sensors, touch sensors, etc., but are not limited to these. The sensor unit may be included in at least one of the main body 110, the transport unit 120, and the handpiece.

Furthermore, the skin treatment device 100 may include a communication unit 220. The communication unit 220 may be configured to enable the skin treatment device 100 to communicate wirelessly or via a wired connection with internal modules or external devices. External devices may include external servers and user terminals. User terminals may include PCs, smartphones, tablets, or wearable devices. The communication unit 220 may include wired/wireless communication modules for network access. Wireless communication technologies that may be used include, for example, WLAN (Wireless LAN) (Wi-Fi), Wibro (Wireless broadband), Wimax (World Interoperability for Microwave Access), HSDPA (High Speed Downlink Packet Access), etc. Wired communication technologies may include, for example, XDSL (Digital Subscriber Line), FTTH (Fiber to the Home), and PLC (Power Line Communication). Furthermore, the network connection unit may include a short-range communication module, enabling it to transmit and receive data with any device/terminal located at close range. For example, short-range communication technologies such as Bluetooth, RFID (Radio Frequency Identification), IrDA (Infrared Data Association), UWB (Ultra-Wideband), and ZigBee may be used, but are not limited to these.

The skin treatment device 100 may include a memory 230. The control unit 200 can execute instructions stored in the memory 230. The memory 230 may be included within the control unit 200 or located externally to the control unit 200. The memory 230 can store various information related to the skin treatment device 100. For example, the memory 230 may include information related to the operating method of the handpiece 130, captured images, and user authentication information, among other things.

The memory 230 may be implemented via a non-volatile storage medium capable of continuously storing arbitrary data. For example, the memory 230 may include storage devices based on flash memory and/or battery-backed memory, as well as disk, optical disk, and magneto-optical storage devices, without limitation. Memory 230 may refer to volatile storage devices, such as random access memory (RAM) like dynamic random access memory (DRAM) or static random access memory (SRAM), which are the primary storage devices directly accessed by the processor and whose stored information is instantly erased when power is turned off, but is not limited to these. This memory 230 may be operated by the control unit 200.

Furthermore, the skin treatment device 100 may additionally include an operation unit that provides an interface for operating the skin treatment device 100. The operation unit may include an output unit 240 and an input unit 250.

The output unit 240 can output information related to treatment, patient information, information related to handpiece control, and information related to the status of the main unit as sound and video under the control of the control unit 200. The output unit 240 may include a speaker or a display. The output unit 240 can output medical images generated by the control unit 200. The output unit 240 can output information necessary for the user to operate the skin treatment device 100, such as a user interface (UI), user information, or target object information. Examples of the output unit 240 include a speaker, printer, CRT display, LCD display, PDP display, OLED display, FED display, LED display, VFD display, DLP display, FPD display, 3D display, transparent display, and may include various other output devices within the scope obvious to those skilled in the art.

The output unit 240 can display information related to the treatment. Information related to treatment may include at least one of the treatment method to be provided to the patient, the treatment time, and information related to the patient's disease. The treatment method may include at least one of ultrasound therapy, RF (Radio Frequency) therapy, and drug injection. The output unit 240 may include information related to the patient. Information related to the patient may include at least one of the patient's age, gender, treatment period, and information related to the disease. Information related to handpiece control may include at least one of the buttons for controlling the handpiece, the operation cycle, the type of base unit currently coupled to the handpiece, the intensity of the energy used for treatment, and the frequency. Information related to the main unit's status may include at least one of the battery status of the main unit, the power connection status, and the error status.

Furthermore, the output unit 240 may output information related to the control of the handpiece 130. For example, when the handpiece 130 is operating, the control unit 200 may control the output unit 240 to output sound or light so that people nearby can recognize that the handpiece is in operation.

The skin treatment device 100 may be connected to the workstation via wired or wireless means. The workstation may also be located in a physically separate space from the skin treatment device 100.

The workstation may include a storage server. The storage server may store at least one of patient information, treatment information, and user (medical practitioner) information. The workstation may include a review device. The review device can receive medical images from the storage server based on user commands and display the medical images. The workstation and the skin treatment device 100 can transmit, store, process, and output data according to the DICOM (Digital Imaging and Communications in Medicine) standard. Additionally, the workstation may include a PACS (Picture Archiving and Communication System).

The workstation may include an output unit, an input unit, and a control unit. The output unit and input unit provide an interface for the user to operate the workstation and the skin treatment device 100. The control unit of the workstation may control the workstation and the skin treatment device 100.

The skin treatment device 100 can be controlled via the workstation and can also be controlled by the control unit 200 included within the skin treatment device 100. Therefore, the user can control the skin treatment device 100 via the workstation or control the skin treatment device 100 via the operation unit and control unit 200 included in the skin treatment device 100. In other words, the user can either remotely control the skin treatment device 100 via the workstation or directly control the skin treatment device 100.

The control unit of the workstation and the control unit 200 of the skin treatment device 100 may be separate, but this is not limited thereto. The control unit of the workstation and the control unit 200 of the skin treatment device 100 may be implemented as a single integrated control unit, and the integrated control unit may be included in only one of the workstation and the skin treatment device 100. Hereinafter, the control unit 200 may refer to the control unit of the workstation and/or the control unit of the skin treatment device 100.

The output unit and input unit of the workstation and the output unit 240 and input unit 250 of the skin treatment device 100 may each provide an interface to the user for operating the skin treatment device 100. The workstation and the skin treatment device 100 may each include output and input units, but are not limited thereto. An output or input unit may be implemented in only one of the workstation and the skin treatment device 100.

Hereinafter, the input unit 250 refers to the input unit of the workstation and/or the input unit of the skin treatment device 100, and the output unit 240 refers to the output unit of the workstation and/or the output unit of the skin treatment device 100.

The input unit 250 can receive commands for operating the skin treatment device 100 and various information related to X-ray imaging from the user. The control unit 200 can control or operate the skin treatment device 100 based on the information input to the input unit 250. The input unit 250 may include a joystick, keyboard, mouse, touch screen, capture button, lock release button, voice recognition device, fingerprint recognition device, iris recognition device, and other input devices obvious to those skilled in the art. The user can input commands for operating the handpiece via the input unit 250, which may include switches for such command input.

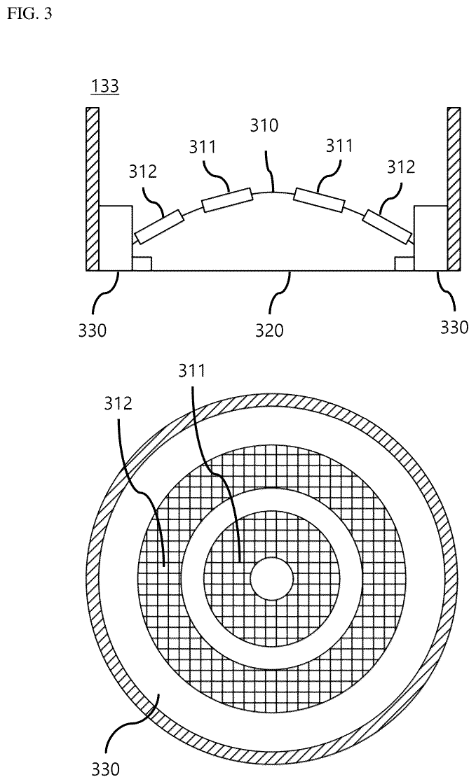

FIG. 3 is a cross-sectional view of the base unit according to an embodiment of the present disclosure.

Referring to FIG. 3, the base unit 133 may include a transducer placement surface 310. The transducer placement surface 310 may have a flat surface relative to the patient's skin.

The base unit 133 may include a skin contact surface 320, which is the surface in contact with the patient's skin. The skin contact surface 320 may be made of an elastic material to help the base unit 133 adhere closely to the skin.

The base unit 133 may include a plurality of ring-shaped transducers 311, 312. The plurality of transducers 311, 312 may be formed on the concave transducer placement surface 310. The plurality of transducers 311, 312 can vibrate at a predetermined treatment frequency to deliver vibrational energy to the patient's skin. The predetermined treatment frequency can be 200 kHz or more and 20 MHz or less. The plurality of transducers 311, 312 may include a first transducer 311 and a second transducer 312 having a larger diameter than the first transducer 311.

The plurality of transducers 311, 312 may have different diameters. The ring-shaped plurality of transducers 311, 312 may form concentric circles. The plurality of transducers 311, 312 may form plural focal points vertically beneath the skin from the center of the concentric circles, enabling simultaneous treatment of different layers beneath the skin. The depth of the focal points formed beneath the skin by the plurality of transducers 311, 312 may be proportional to the diameter of each respective the plurality of transducers 311, 312. The longer the diameter of a transducer 311, 312, the deeper the focal point may be. The second transducer 312 may have a larger diameter than the first transducer 311. Therefore, the depth of the focus of the second transducer 312 may be greater than the depth of the focus of the first transducer 311. Here, the depth of focus may refer to the distance from the center of the skin contact surface 320 to the point where vibrational energy is concentrated. The skin treatment device 100 may enable more effective treatment by simultaneously treating different layers beneath the patient's skin. On the transducer placement surface 310, the plurality of transducers 311, 312 may be formed in contact such that the second transducer 312, having a larger diameter, wraps around the circumference of the first transducer 311. Alternatively, the plurality of transducers 311, 312 may be spaced apart and non-contacting.

The base unit 133 may include a thermoelectric element 330). The thermoelectric element 330 may be formed on the concave transducer placement surface 310. The thermoelectric element 330 may be formed along the outer periphery of the plurality of transducers 311, 312. The thermoelectric element 330 may be formed along the edge of the transducer placement surface 310. The thermoelectric element 330 may contact at least one of the transducer placement surface 310, the skin contact surface 320, and the plurality of transducers 311, 312. The thermoelectric element 330 may cool at least one of the transducer placement surface 310, the skin contact surface 320, and the plurality of transducers 311, 312. The cooling surface of the thermoelectric element 330 may contact at least one of the plurality of transducers 311, 312. The thermoelectric element 330 may cool at least one of the plurality of transducers 311, 312 it contacts to prevent the patient's skin surface in contact with one side of the handpiece 130 from being burned. The thermoelectric element may also be formed directly on the base unit 133 along its inner wall. The cooling surface of the thermoelectric element 330 may face the skin. The cooling surface of the thermoelectric element 330 may contact the skin together with the skin contact surface 320. The thermoelectric element 330 may cool the cooling surface contacting the skin to prevent the patient's skin surface from being burned.

The plurality of transducers 311, 312 may radiate vibrational energy simultaneously or sequentially based on user input regarding the treatment area. The skin treatment device 100 may store skin information regarding the treatment area input by the user. For example, the handpiece 130 may include a sensor for measuring skin thickness. The skin treatment device 100 may determine the skin thickness of the area to be treated based on at least one of the skin thickness input from the user and the skin thickness measured by the sensor. If the skin thickness of the area to be treated is above a certain thickness, the first transducer 311 and the second transducer 312 may simultaneously radiate vibrational energy. If the skin thickness is below a certain thickness, only one of the first transducer 311 and the second transducer 312 may radiate vibrational energy. The skin treatment device 100 can control the operation of the plurality of transducers 311, 312 according to the skin thickness to avoid treating near the nerve tissue, thereby reducing patient discomfort during the procedure.

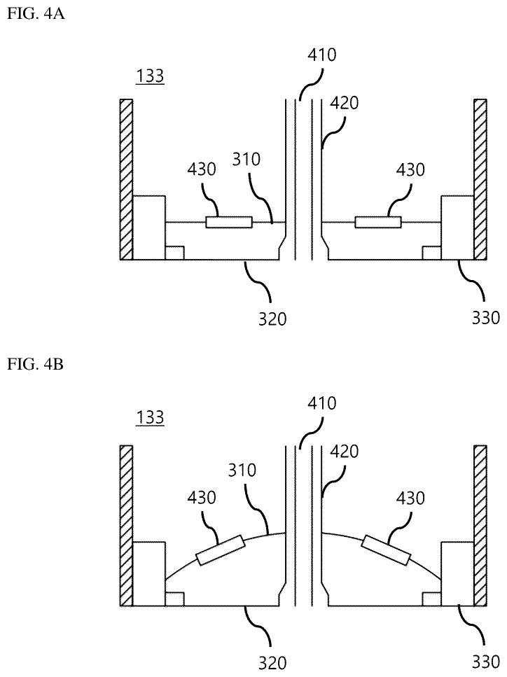

FIGS. 4A and 4B are cross-sectional views of the base unit according to an embodiment of the present disclosure.

The handpiece 130 may include a drug injection unit 410 for injecting drugs into the patient's skin. The drug injection unit 410 may be coupled to a drug supply unit via a rubber tube. The drug injection unit 410 may be located at the center of the base unit 133. The drug injection unit 410 may pass through the transducer placement surface 310 and the skin contact surface 320. One side of the drug injection unit 410 may contact the patient's skin. The drug injection unit 410 may spray the drug supplied from the drug supply unit at the point where it contacts the patient's skin.

The handpiece 130 may include a skin suction unit 420 that sucks the patient's skin. The skin suction unit 420 may be formed along the outer periphery of the drug injection unit 410. The skin suction unit 420 and the drug injection unit 410 may form concentric circles. The skin suction unit 420 may surround the circumference of the drug injection unit 410. For example, it may be configured such that a tube forming the drug injection unit 410 is formed inside a tube forming the skin suction unit 420.

Referring to FIG. 4A, the transducer placement surface 310 may have a flat surface relative to the patient's skin. The transducer placement surface 310 may include a ring-shaped transducer 430 that vibrates at a predetermined treatment frequency to deliver vibrational energy to the patient's skin. The transducer 430 may be formed along the outer periphery of the skin suction unit 420. The transducer 430 included in the transducer placement surface 310 having a flat surface can assist in the rapid absorption of the drug from the drug injection unit 410 into the skin. Furthermore, the transducer 430 included in the transducer placement surface 310 having a flat surface can emit vibrational energy to treat the skin. For example, constructive interference of vibrations emitted from the transducer can form large vibrational energy at specific locations beneath the skin to create lesions.

Referring to FIG. 4B, the transducer placement surface 310 may have a concave surface relative to the patient's skin. With a concave transducer placement surface 310, the transducer 430 can emit vibrational energy perpendicular to the slope direction of the concave surface. Therefore, the transducer 430 can form a focused beam along the central axis of the transducer placement surface 310.

At least one of the drug injection unit 410 and the skin suction unit 420 may form a concentric circle with at least one of the transducer 430 and the thermoelectric element 330. By forming concentric circles with the transducer 430, the drug injection unit 410 and skin suction unit 420 position the area where the transducer 430 generates the focus and the area where the drug injection unit 410 injects the drug along the same axis, enabling precise drug injection into the treatment site.

The skin suction unit 420 can create negative pressure by suctioning the patient's skin. At least one of the main body 110 or the handpiece 130 may include a compressor. If the compressor is formed in the main body 110, it can be coupled to the skin suction unit 420 through a tube included in the cable 131. The skin suction unit 420 can secure the handpiece 130 in close contact with the area to be treated by suctioning the skin during the procedure. This minimizes the impact of movements that could affect the procedure, such as hand tremors of the user performing the procedure with the handpiece 130 or patient movement, enabling a more precise procedure.

The main body 110 or handpiece 130 may include a pressure sensor for measuring the pressure formed inside the skin suction unit 420. Based on the pressure sensor, the control unit 200 can measure the pressure formed inside the skin suction unit 420. The control unit 200 can control the drug injection unit 410 to stop drug injection when the pressure formed inside the skin suction unit 420 is equal to or greater than the critical pressure. Furthermore, the control unit 200 can control the transducer 430 to not emit vibration energy when the pressure formed inside the skin suction unit 420 is equal to or greater than a critical pressure. The critical pressure may be a pressure value sufficient to fix the treatment site by suctioning the skin. The critical pressure may be a predetermined value. The control unit 200 can control to perform at least one of stopping drug injection and stopping vibration energy emission when the pressure formed inside the skin suction unit 420 is equal to or greater than a critical pressure, during the procedure. The control unit 200 can control to transmit an audible warning and a warning message to the output unit 240 to notify the user to re-press the handpiece 130 against the skin.

The control unit 200 may control the drug injection unit 410 to inject the drug when the pressure in the skin suction unit 420 is below the critical pressure. The control unit 200 may control the transducer 430 to emit vibrational energy when the pressure in the skin suction unit 420 is below the critical pressure. The control unit 200 can ensure safe treatment by controlling the emission of vibrational energy only when the handpiece is in close contact with the skin.

However, this is not limited to this, and the handpiece 130 may include a proximity sensor. The proximity sensor may be a sensor that measures the distance between the handpiece and the patient's skin, or determines whether the handpiece and the patient's skin are sufficiently close to provide treatment. If the proximity sensor indicates that the distance between the patient's skin and the handpiece 130 is greater than a predetermined critical distance, the control unit 200 may control the drug injection unit 410 to stop drug injection. Furthermore, if the proximity sensor indicates that the distance between the patient's skin and the handpiece 130 is less than a predetermined threshold distance, the control unit 200 may control the drug injection unit 410 to inject the drug.

The handpiece 130 may include a filter for filtering drugs and waste materials sucked in by the skin suction unit 420. The filter may be positioned between the compressor and the skin suction unit 420. The filter may be replaceable by the user. The filter may allow air between the skin suction unit 420 and the compressor to pass through while preventing the passage of drug and waste. The filter may prevent drug and waste sucked by the skin suction unit 420 from entering the compressor and the interior of the handpiece. The filter included in the handpiece 130 may facilitate maintenance of the handpiece 130. The user can maintain the cleanliness of the handpiece 130 simply by replacing the filter.

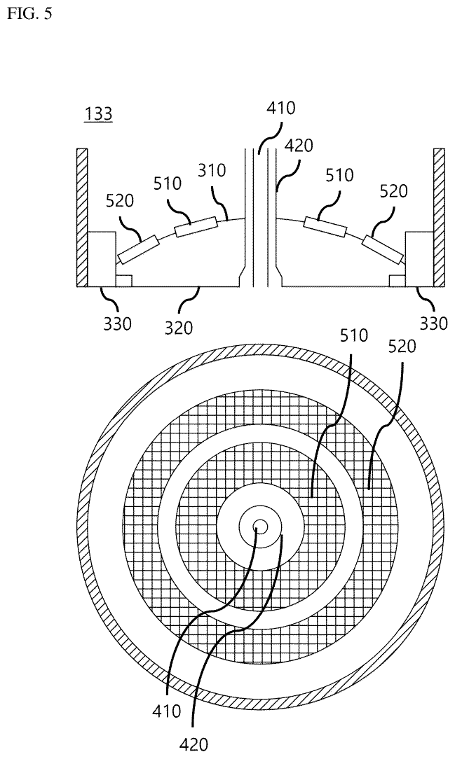

FIG. 5 is a cross-sectional view of the base unit according to an embodiment of the present disclosure.

Referring to FIG. 5, the handpiece 130 may simultaneously incorporate a plurality of transducers 510, 520, a skin suction unit 420, a drug injection unit 410, and a thermoelectric element 330 may be simultaneously formed. The base unit 133 may include a transducer placement surface 310 having a concave surface relative to the patient's skin. The transducer placement surface 310 may be located on the upper side of the skin contact surface 320. The plurality of transducers 510, 520 in FIG. 5 may include the same configuration as the plurality of transducers 311, 312 in FIG. 3. The drug injection unit 410 and skin suction unit 420 of FIG. 5 may include the same configuration as the drug injection unit 410 and skin suction unit 420 of FIGS. 4A and 4B.

The plurality of transducers 510, 520, skin suction unit 420, drug injection unit 410, and thermoelectric element 330 may form concentric circles. By forming concentric circles with each other, the plurality of transducers 510, 520 can form multiple focal points above and below the center axis of the transducer placement surface 310. The multiple focal points of the plurality of transducers 510, 520 can simultaneously and intensively treat different layers beneath the skin. The drug injection unit 410 and skin suction unit 420 form concentric circles with the plurality of transducers 510, 520. This alignment ensures the area where the plurality of transducers 510, 520 generate focal points coincides with the area where the drug injection unit 410 injects the drug, enabling precise drug injection at the treatment site.

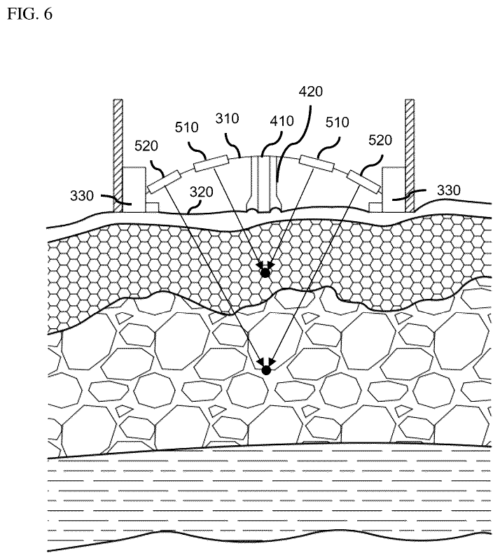

FIG. 6 is a drawing illustrating a handpiece according to an embodiment of the present disclosure.

Hereinafter, with reference to FIG. 5 and FIG. 6, the process by which the skin treatment device provides ultrasonic therapy according to various embodiments of the present disclosure will be described.

FIG. 7 is a flowchart illustrating the operation of the skin treatment device according to an embodiment of the present disclosure.

The control unit 200 may perform a step 710 of acquiring the cumulative shot count of the plurality of transducers 510, 520. The cumulative shot count may be a value accumulated from the number of shots delivered to the patient by at least one of the first transducer 510 and the second transducer 520 since the procedure began. The cumulative shot count may be obtained for the first transducer 510 and the second transducer 520 respectively. The control unit 200 may control at least one of the first transducer 510 and the second transducer 520 based on the cumulative shot count of the first transducer 510. The control unit 200 may control at least one of the first transducer 510 and the second transducer 520 based on the cumulative shot count of the second transducer 520. The control unit 200 may measure time elapsed since the procedure began. The start of the procedure may be when at least one of the plurality of transducers 510, 520 begins emitting vibrational energy. The control unit 200 may acquire, for each shot delivered after the procedure begins, the time at which that shot was delivered.

The control unit 200 may perform a step 720 of obtaining the skin temperature for the cumulative number of shots based on at least one of a first table and a first function that correlates the cumulative number of shots to the skin temperature. The control unit 200 may obtain the skin temperature by applying the cumulative number of shots to at least one of the first table and the first function. The first table may include a first-first table and a first-second table. The first function may include a first-first function and a first-second function. The control unit 200 may store at least one of the first-first table and the first-first function, which map the cumulative shot count to the patient's skin temperature. At least one of the first-first table and the first-first function may be based on the following function. For example, the first-first table may be a table mapping values of T1 to s and T0 based on the following equation.

$$A 1 * s + T 0 = T 1$$

However, this is not limited to this, and at least one of the first-first table and First-first function may be based on the following function.

$$A 1 * log ( s ) + T 0 = T 1$$

Here, s may be the cumulative number of shots. T0 may be the initial skin temperature. At the start of the procedure, T0 may be determined as at least one of a predetermined skin temperature or ambient temperature. T1 may be the current skin temperature. Past T1 values may be used as T0 to determine the current skin temperature at the next time point. A1 is the skin heating rate due to the transducer and may be a predetermined positive real number. A1 may be obtained via the following equation.

$$A 1 = k / V + b$$

where V may be the voltage applied to the thermoelectric element 330. k may be a predetermined positive real number. b may be a predetermined constant. A1 may be inversely proportional to the voltage applied to the thermoelectric element 330.

However, this is not limited to this, and A1 may be obtained via the following equation:

$$A 1 = k * V + b$$

Here, k may be a predetermined negative real number. The value of A1 may decrease as the voltage applied to the thermoelectric element 330 increases.

As the cumulative shot count increases, the patient's skin temperature may rise. The control unit 200 can obtain the skin temperature by correlating the cumulative shot count with at least one of the first-first table and First-first function. The control unit 200 may store at least one of the first-second table and First-second function, which correlate the patient's skin temperature with natural cooling. If the procedure is stopped midway, the control unit 200 may acquire the time the procedure was stopped and the time it was resumed. The control unit 200 may acquire the duration the procedure was stopped. The duration the procedure was stopped may be the time elapsed from when the procedure was stopped until it was resumed. At least one of the first-second table and the first-second function may be based on the following function:

$$B 1 * t + T 0 = T 1$$

However, this is not limited thereto, and at least one of the first-second table and the first-second function may be based on the following function:

$$B 1 / t + T 0 = T 1$$

Here, B1 may be a predetermined cooling slope due to natural cooling. B1 may be a predetermined negative real number. T0 may be the skin temperature at the time the procedure was stopped. t may be the duration the procedure was stopped. T1 may be the skin temperature at the time the procedure was resumed.

The control unit 200 can obtain the patient's skin temperature by correlating the duration the procedure was stopped with at least one of the first-second table and the first-second function. The longer the procedure is stopped, the lower the skin temperature may become. When the procedure resumes, the control unit 200 can use the skin temperature obtained via at least one of the first-second table and first-second function as the initial skin temperature for at least one of the first-first table and first-first function.

Step 720 is automatically performed at predetermined intervals during the procedure to automatically acquire the skin temperature of the patient in contact with the handpiece 130. The skin treatment device 100 can display the acquired skin temperature in real time on the display of the output unit 240.

The control unit 200 may perform step 730 of determining the treatment shot count based on the skin temperature relative to the cumulative shot count. The treatment shot count may be the number of shots to be delivered to the patient in the future by at least one of the first transducer 510 and the second transducer 520. The treatment shot count may be the predetermined number of shots per hour for at least one of the first transducer 510 and the second transducer 520. The treatment shot count may be determined separately for each of the first transducer 510 and the second transducer 520. The control unit 200 may store data of the treatment shot count based on skin temperature. The control unit 200 may adjust the treatment shot count based on the skin temperature acquired in step 720. The higher the skin temperature, the smaller the treatment shot count may be, and the lower the skin temperature, the larger the treatment shot count may be. However, this is not limited to this, and the treatment shot count may be constant regardless of the level of skin temperature.

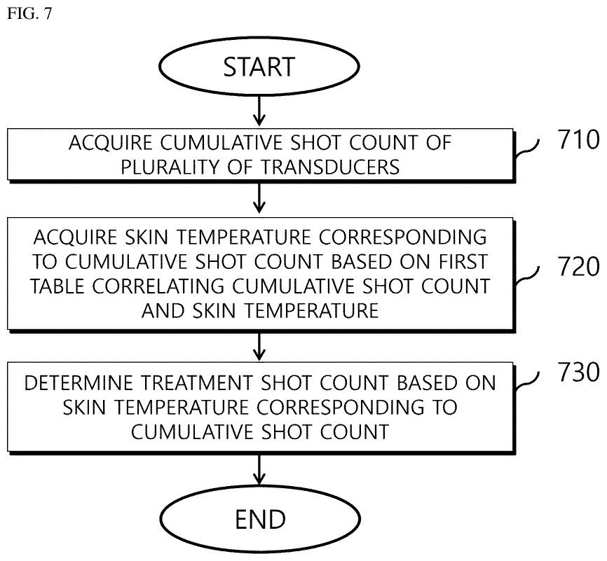

FIG. 8 is a flowchart for explaining the operation of a skin treatment device according to an embodiment of the present disclosure.

The control unit 200 may perform a step 810 of acquiring the cumulative irradiation time of the plurality of transducers 510, 520. The cumulative irradiation time may be the accumulated value of the time during which at least one of the first transducer 510 and the second transducer 520 irradiated ultrasound to the patient after the procedure began. The cumulative irradiation time may be acquired for the first transducer 510 and the second transducer 520 respectively. The control unit 200 may control at least one of the first transducer 510 and the second transducer 520 based on the cumulative irradiation time of the first transducer 510. The control unit 200 can control at least one of the first transducer 510 and the second transducer 520 based on the cumulative irradiation time of the second transducer 520. The control unit 200 can measure time from the start of the procedure. The control unit 200 can acquire the time when ultrasonic radiation began and the time when radiation ended.

The control unit 200 may perform a step 820 of obtaining skin temperature for the cumulative irradiation time based on at least one of a second table and a second function that correlates the cumulative irradiation time with skin temperature. The control unit 200 may obtain skin temperature by applying the cumulative irradiation time to at least one of the second table and the second function. The second table may include a second-first table and a second-second table. The second function may include a second-first function and a second-second function. The control unit 200 may store at least one of the second-first table and the second-first function, which correlate cumulative irradiation time with skin temperature. At least one of the second-first table and the second-first function may be based on the following function:

$$A 2 * r + T 0 = T 1$$

However, this is not limited thereto, and at least one of the second-first table and the second-first function may be based on the following function:

$$A 2 * log ( r ) + T 0 = T 1$$

Here, r may be the cumulative irradiation time. T0 may be the initial skin temperature. T1 may be the current skin temperature. A2 is the skin heating rate due to the transducer and may be a predetermined positive real number. A2 can be obtained via the following equation:

$$A 2 = k / V + b$$

Here, V may be the voltage applied to the thermoelectric element 330. k may be a predetermined positive real number. b may be a predetermined constant. A2 may be inversely proportional to the voltage applied to the thermoelectric element 330.

However, this is not limited to this, and A2 can be calculated using the following equation.

$$A 2 = k * V + b$$

Here, k can be a predetermined negative real number. The value of A2 can decrease as the voltage applied to the thermoelectric element 330 increases.

As the cumulative irradiation time increases, the patient's skin temperature may rise. The control unit 200 can obtain the skin temperature by correlating the cumulative irradiation time with at least one of the second-first table and the second-first function.

The control unit 200 may store at least one of a second-second table and a second-second function that corresponds to the patient's skin temperature due to natural cooling. If the procedure is stopped midway, the control unit 200 may obtain the time when the procedure was stopped and the time when it was resumed. The control unit 200 may obtain the duration the procedure was stopped. The duration of the procedure interruption may be the time elapsed from the time the procedure was interrupted until the time it was resumed. At least one of the second-second table and the second-second function may be based on the following function:

$$B 2 * t + T 0 = T 1$$

However, this is not limited thereto, and at least one of the second-second table and the second-second function may be based on the following function:

$$B 2 / t + T 0 = T 1$$

Here, B2 may be a predetermined cooling slope due to natural cooling. B2 may be a predetermined negative real number. T0 may be the skin temperature at the time the procedure was stopped. t may be the time the procedure was stopped. T1 may be the skin temperature at the time the procedure was resumed.

The control unit 200 can obtain the patient's skin temperature by correlating the time the procedure was stopped with at least one of the second-second table and the second-second function. The longer the procedure is stopped, the lower the skin temperature may become. When the procedure resumes, the control unit 200 can use the skin temperature obtained via at least one of the second-second table and second-second function as the initial skin temperature for at least one of the second-first table and second-first function.

Step 820 is automatically performed at predetermined intervals during the procedure to automatically acquire the skin temperature of the patient in contact with the handpiece 130. The skin treatment device 100 can display the acquired skin temperature in real time on the display of the output unit 240.

The control unit 200 may perform a step of determining the treatment irradiation time based on the skin temperature relative to the cumulative irradiation time. The treatment irradiation time may be the irradiation time of ultrasound to be provided to the patient by the first transducer 510 and the second transducer 520. The treatment irradiation time may be determined separately for the first transducer 510 and the second transducer 520. The control unit 200 may store treatment irradiation time data corresponding to skin temperatures. The control unit 200 may adjust the treatment irradiation time based on the skin temperature acquired in step 820. The treatment irradiation time may decrease as the skin temperature increases, and may increase as the skin temperature decreases. However, this is not limited to this, and the treatment irradiation time may remain constant regardless of the skin temperature.

The steps (710, 720, 730) in FIG. 7 and the steps (810, 820, 830) in FIG. 8 can be performed individually or simultaneously. When performed simultaneously, the control unit 200 can acquire the skin temperature obtained in step 720 of FIG. 7 and the skin temperature obtained in step 820 of FIG. 8. The control unit 200 can obtain a representative skin temperature using the skin temperature from step 720 and the skin temperature from step 820. The representative skin temperature can be one of the average, median, maximum, or minimum values of the skin temperatures from step 720 and step 820. The control unit 200 can display the representative skin temperature to the output unit 240. The control unit 200 can use the representative skin temperature to perform step 730 and step 830 to obtain the treatment shot count and the treatment irradiation time.

The control unit 200 may include information regarding a sufficient time for the plurality of transducers 510, 520 to cool naturally. Upon elapse of that time, the control unit 200 may initialize the cumulative shot count and cumulative irradiation time.

The control unit 200 may automatically regulate the temperature of the thermoelectric element 330 by controlling the voltage applied to the thermoelectric element 330. Due to the Peltier effect, the higher the voltage applied to the thermoelectric element 330, the lower the temperature of the cooling surface of the thermoelectric element 330 can become. The lower the voltage applied to the thermoelectric element 330, the higher the temperature of the cooling surface of the thermoelectric element 330 can become.

The control unit 200 can determine the voltage to apply to the thermoelectric element 330 based on the skin temperature obtained from at least one of the first table, first function, second table, and second function and at least one of the third table and third function that maps skin temperature to the voltage applied to the thermoelectric element 330. However, this is not limited thereto, and the control unit 200 may also determine the voltage to be applied to the thermoelectric element 330 based on the skin temperature directly measured and obtained and at least one of the third table and the third function.

The third table may include the third-first table and the third-second table. The third function may include the third-first function and the third-second function. The control unit 200 may store at least one of the third-first table and the third-first function, which map skin temperature to the voltage applied to the thermoelectric element 330. At least one of the third-first table and the third-first function may be based on the following function:

$$C 1 * T + V 0 = V 1$$

However, this is not limited thereto, and at least one of the third-first table and the third-first function may be based on the following function:

$$C 1 * log ( T ) + V 0 = V 1$$

Here, T may be the skin temperature. V0 may be the initial voltage. V1 may be the voltage applied by the thermoelectric element 330. C1 is the heating slope of the skin temperature and may be a predetermined positive real number.

Furthermore, at least one of the third-first table and third-first function may be based on the following function.

$$C 1 / T + V limit = V 1$$

Here, C1 is the heating slope of the skin temperature, which may be a predetermined negative real number. T may be the skin temperature. Vlimit may be a predetermined constant. V1 may be the voltage applied to the thermoelectric element 330.

The voltage applied to the thermoelectric element 330 may increase as the skin temperature rises. The first table, first function, second table, and second function may include skin temperature data considering the cooling of the thermoelectric element 330. Therefore, the control unit 200 may determine the voltage to be applied to the thermoelectric element 330 by applying the skin temperature obtained from at least one of the first table, first function, second table, and second function to at least one of the third-first table and third-first function. As the skin temperature rises, the voltage applied to the thermoelectric element 330 also increases, preventing the skin temperature from exceeding a predetermined threshold. Consequently, the patient's skin may not sustain burns.

The control unit 200 may store at least one of third-second table and Third-second function, which correspond the voltage applied to the thermoelectric element 330 to natural cooling. If the procedure is stopped midway, the control unit 200 may acquire the time the procedure was stopped and the time it was resumed. The control unit 200 may acquire the duration of the procedure's suspension. The duration of the suspension may be the time elapsed from the time the procedure was suspended until the time it was resumed. At least one of the third-second table and the third-second function may be based on the following function:

$$C 2 * t + V 1 = V 2$$

Here, C2 is the cooling slope due to natural cooling and may be a predetermined negative real number. V1 may be the initial voltage of the thermoelectric element 330. V2 may be the voltage to be applied to the thermoelectric element 330. t may be the time the procedure was suspended.

However, this is not limited thereto, and at least one of the third-second table and Third-second function may be based on the following function.

$$C 2 / ( t + a ) + V 1 - C 2 / a = V 2$$

Here, C2 is the cooling slope due to natural cooling, which may be a predetermined positive real number. t may be the time during which the procedure is stopped. a is a predetermined constant, which may be a predetermined positive real number. V1 may be the initial voltage of the thermoelectric element 330. V2 may be the voltage applied to the thermoelectric element 330.

As the time t during which the procedure is stopped increases, the voltage V2 applied to the thermoelectric element 330 may decrease.

As in the embodiments of FIGS. 7 and 8, the skin treatment device 100 can acquire skin temperature without including a temperature sensor. This enables the skin treatment device 100 to achieve miniaturization of the handpiece 130. Furthermore, not using a temperature sensor reduces the cost of the skin treatment device 100. The skin treatment device 100 can achieve the same skin improvement effects and stability as a device equipped with a conventional temperature sensor by automatically controlling the plurality of transducers 510, 520 and thermoelectric element 330 without using a temperature sensor.

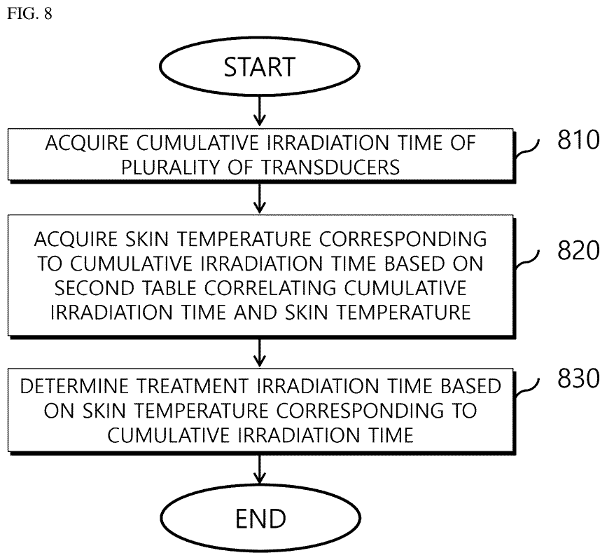

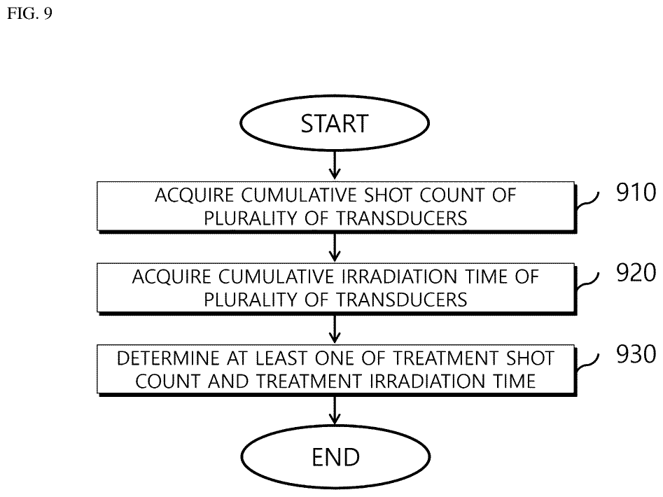

FIG. 9 is a flowchart illustrating the operation of the skin treatment device according to an embodiment of the present disclosure.

The control unit 200 may perform a step 910 of acquiring the cumulative shot count of the plurality of transducers 510, 520. The control unit 200 may perform a step 920 of acquiring the cumulative irradiation time of the plurality of transducers 510, 520. The control unit 200 may perform a step 930 of determining at least one of the treatment shot count and the treatment irradiation time based on at least one of the cumulative shot count and the cumulative irradiation time. The control unit 200 may store at least one of a fourth table and a fourth function that corresponds at least one of the cumulative shot count and the cumulative irradiation time to at least one of the treatment shot count and the treatment irradiation time. Step 930 may be performed based on at least one of the fourth table and the fourth function. The fourth table may include fourth-first table, fourth-second table, fourth-third table, and fourth-fourth table. The fourth function may include the fourth-first function, fourth-second function, fourth-third function, and fourth-fourth function.

The control unit 200 may store at least one of a fourth-first table and a fourth-first function that maps the cumulative shot count to the treatment shot count. At least one of the fourth-first table and fourth-first function may be based on the following function:

$$A 3 / s = s 1$$

Here, s may be the cumulative shot count. s1 may be the treatment shot count. A3 is the heating slope, which may be a predetermined positive real number.

However, this is not limited thereto, and at least one of the fourth-first table and the fourth-first function may be based on the following function:

$$A 3 * s + s 0 = s 1$$

Here, s may be the cumulative shot count. s1 may be the treatment shot count. A3 is the heating slope, which may be a predetermined negative real number. s0 may be the initial treatment shot count. As the cumulative shot count s increases, the treatment shot count s1 may decrease.

The control unit 200 may store at least one of a fourth-second table and a fourth-second function that maps cumulative irradiation time to treatment irradiation time. At least one of the fourth-second table and the fourth-second function may be based on the following function.

$$A 3 / r = r 1$$

Here, r may be the cumulative irradiation time. r1 may be the treatment irradiation time. A3 is the heating slope, which may be a predetermined positive real number.

However, this is not limited thereto, and at least one of the fourth-first table and fourth-first function may be based on the following function.

$$A 3 * r + r 0 = r 1$$

Here, r may be the cumulative irradiation time. r1 may be the treatment irradiation time. A3 is the heating slope, which may be a predetermined negative real number. r0 may be the initial treatment irradiation time. As the cumulative irradiation time r increases, the treatment irradiation time r1 may decrease.

The control unit 200 can obtain at least one of the treatment shot count and treatment irradiation time by correlating at least one of the cumulative shot count and cumulative irradiation time with at least one of the fourth-first table, fourth-first function, fourth-second table, and Fourth-second function. Therefore, the control unit 200 can automatically adjust the treatment shot count and treatment irradiation time without acquiring skin temperature.

If the procedure is temporarily paused, the plurality of transducers 510, 520 and skin temperature may naturally cool down over time. The control unit 200 can acquire duration the procedure was stopped. The control unit 200 may store at least one of fourth-third table and Fourth-third function. fourth-third table and Fourth-third function may include the treatment shot count corresponding to the duration of the procedure was stopped. The control unit 200 may store at least one of fourth-fourth table and Fourth-fourth function. The fourth-fourth table and fourth-fourth function may include the treatment irradiation time corresponding to the duration of the procedure was stopped. In a subsequently resumed procedure, the control unit 200 may use the fourth-first table, fourth-first function, fourth-second table and fourth-second function to provide treatment using at least one of the treatment shot count and the treatment irradiation time corresponding to at least one of the treatment shot count and the treatment irradiation time obtained.

At least one of the fourth-third table and the fourth-third function may be based on the following function:

$$B 3 * t + s 0 = s 1$$

However, this is not limited thereto, and at least one of the fourth-third table and fourth-third function may be based on the following function:

$$B 3 * log ( t ) + s 0 = s 1$$

where B3 is the cooling slope due to natural cooling, which may be a predetermined positive real number; t may be the time at which the procedure was stopped. s0 may be the initial treatment shot count. s1 may be the current treatment shot count.

At least one of fourth-fourth table and fourth-fourth function may be based on the following function.

$$B 3 * t + r 0 = r 1$$

However, this is not limited to this, and at least one of the fourth-fourth table and the fourth-fourth function may be based on the following function.

$$B 3 * log ( t ) + r 0 = r 1$$

Here, B3 is the cooling slope due to natural cooling and may be a predetermined positive real number. t may be the time the procedure was suspended. r0 may be the initial treatment irradiation time. r1 may be the current treatment irradiation time.

As the time at which the procedure is stopped increases, at least one of the treatment shot count s1 and the treatment irradiation time r1 may increase.

In the embodiment of FIG. 9, the skin treatment device 100 may acquire at least one of the treatment shot count and the treatment irradiation time without acquiring the skin temperature. By not acquiring skin temperature, the skin treatment device 100 may achieve a faster processing speed for providing at least one of the treatment shot count and the treatment irradiation time.

According to various embodiments of the present disclosure, control of the thermoelectric element 330 may be performed in conjunction with FIGS. 7 through 9. However, without limitation, control of the thermoelectric element 330 may be performed without implementing FIGS. 7 through 9, allowing the skin treatment device to maintain the temperature of the skin contact surface 320 on the handpiece within a predetermined threshold temperature to prevent the patient's skin surface from being burned. The following describes a method for controlling the thermoelectric element 330.

The control unit 200 can automatically regulate the temperature of the thermoelectric element 330 by controlling the voltage applied to it. As the cumulative shot count and cumulative irradiation time increase, the voltage applied to the thermoelectric element 330 can increase. For example, the control unit 200 can determine the voltage to apply to the thermoelectric element 330 based on the cumulative shot count and cumulative irradiation time. At least one of the third table and the third function may include data regarding the voltage to apply to the thermoelectric element 330 corresponding to at least one of the cumulative shot count and cumulative irradiation time.

The control unit 200 may determine the voltage to be applied to the thermoelectric element 330 by applying at least one of the cumulative shot count obtained in step 910 of FIG. 9 and the cumulative irradiation time obtained in step 920 of FIG. 9 to at least one of the third table and the third function during treatment period.

The third table may include the third-third table, third-fourth table, and third-fifth table. The third function may include the third-third function, third-fourth function, and third-fifth function.

The control unit 200 can determine the voltage to be applied to the thermoelectric element 330 based on at least one of the third-third table and third-third function, which correspond the cumulative shot count to the voltage applied to the thermoelectric element 330. At least one of the third-third table and third-third function can be based on the following function:

$$C 3 * s + V 0 = V 1$$

However, this is not limited thereto, and at least one of the third-third table and the third-third function may be based on the following function:

$$C 3 * log ( s ) + V 0 = V 1$$

Here, C3 is the skin heating slope due to the transducer and may be a predetermined positive real number. s may be the cumulative shot count. V0 may be the initial voltage of the thermoelectric element 330. V1 may be the voltage to be applied to the thermoelectric element 330.

Furthermore, at least one of third-third table and third-third function may be based on the following function.

$$C 3 / s + Vlimit = V 1$$

Here, C3 is the skin heating slope caused by the transducer, which may be a predetermined negative real number. s may be the cumulative shot count. Vlimit may be a predetermined constant. V1 may be the voltage applied to the thermoelectric element 330.

As the cumulative shot count s increases, the voltage V1 applied to the thermoelectric element 330 may increase.

The control unit 200 can determine the voltage to apply to the thermoelectric element 330 based on at least one of the third-fourth table and third-fourth function that correspond the cumulative irradiation time and the voltage applied to the thermoelectric element 330. At least one of the third-fourth table and third-fourth function can be based on the following function:

$$C 3 * r + V 0 = V 1$$

However, this is not limited thereto, and at least one of the third-fourth table and the third-fourth function may be based on the following function:

$$C 3 * log ( r ) + V 0 = V 1$$

Here, C3 is the skin heating slope due to the transducer and may be a predetermined positive real number. r may be the cumulative irradiation time. V0 may be the initial voltage of the thermoelectric element 330. V1 may be the voltage applied to the thermoelectric element 330. The initial voltage of the thermoelectric element may have a predetermined value during the first operation of the skin treatment device 100, and thereafter, V1 may become the initial voltage and be used to determine the next V1.

Furthermore, at least one of the third-fourth table and third-fourth function may be based on the following function.

$$C 3 / r + Vlimit = V 1$$

where C3 is the skin heating rate due to the transducer, which may be a predetermined negative real number. r may be the cumulative irradiation time. Vlimit may be a predetermined constant. V1 may be the voltage applied to the thermoelectric element 330. Based on the function or table as described above, V1 may be obtained at predetermined intervals, and the skin treatment device 100 may control the thermoelectric element based on V1.

As the cumulative irradiation time r increases, the voltage V1 applied to the thermoelectric element 330 may increase.

The control unit 200 may store at least one of the third-fifth table and third-fifth function corresponding to the patient's skin temperature during natural cooling. If the procedure is stopped midway, the control unit 200 may acquire the time the procedure was stopped and the time it was resumed. The control unit 200 may acquire the duration the procedure was stopped. The duration of the procedure interruption may be the time elapsed from the time the procedure was interrupted until the time it was resumed. The control unit 200 may determine the voltage to be applied to the thermoelectric element by applying the treatment stoppage time to at least one of the third-fifth table and third-fifth function during non-treatment period. At least one of the third-fifth table and third-fifth function may be based on the following function.

$$C 4 * t + V 1 = V 2$$

Here, C4 is the cooling slope due to natural cooling and may be a predetermined negative real number. V1 may be the initial voltage of the thermoelectric element 330. V2 may be the voltage to be applied to the thermoelectric element 330. t may be the duration the procedure was stopped.

However, this is not limited thereto, and at least one of the third-fifth table and Third-fifth function may be based on the following function.

$$C 4 / ( t + a ) + V 1 - C 4 / a = V 2$$

Here, C4 is the cooling slope due to natural cooling and may be a predetermined positive real number. t may be the duration the procedure is stopped. a is a predetermined constant and may be a predetermined positive real number. V1 may be the initial voltage of the thermoelectric element 330. V2 may be the voltage to be applied to the thermoelectric element 330.

As the time t at which the procedure is stopped increases, the voltage V2 applied to the thermoelectric element 330 may decrease. Furthermore, if the procedure is restarted, the voltage applied to the thermoelectric element 330 may increase based on at least one of the third-fourth table, the third-fourth function, the third-fourth table, and the third-fourth function. Thus, even if the handpiece does not include a temperature sensor, the handpiece can maintain an appropriate temperature, enabling skin treatment without damaging the patient's skin surface. Furthermore, the absence of a temperature sensor not only reduces the cost of the skin treatment device but also allows for a slim handpiece design, enhancing its aesthetic appeal.

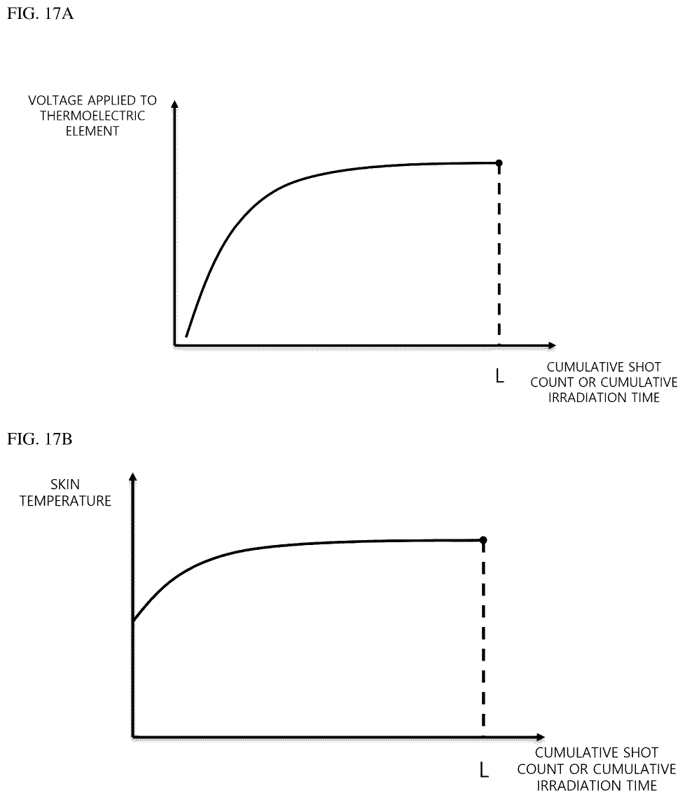

FIGS. 17A and 17B are diagrams illustrating a predetermined function according to an embodiment of the present disclosure.

Referring to FIG. 17A, the voltage applied to the thermoelectric element 330 may increase as the cumulative shot count and cumulative irradiation time increase.

Referring to FIG. 17B, the skin temperature may increase as the cumulative shot count and cumulative irradiation time increase. Here, the skin temperature may be the skin temperature considering the cooling of the thermoelectric element 330.

The control unit 200 may include a limit shot count and a limit irradiation time. The limited shot count and limited irradiation time may be predetermined values (L) of the cumulative shot count and cumulative irradiation time. At the predetermined value (L) of the cumulative shot count and cumulative irradiation time shown in FIG. 17A, a predetermined voltage may be applied to the thermoelectric element 330. Furthermore, if subsequent continuous treatment occurs, the voltage applied to the thermoelectric element 330 may be maintained at a maximum. Of course, if treatment is interrupted, the voltage of the thermoelectric element 330 may decrease.

At the predetermined value (L) of the cumulative shot count and cumulative irradiation time in FIG. 17B, the patient's skin temperature may be lower than the maximum value at which the patient may not sustain a burn. Upon reaching the predetermined value (L) for cumulative shot count and cumulative irradiation time, the heating of the patient's skin surface due to treatment and the cooling of the thermoelectric element reach thermal equilibrium, allowing the patient's skin temperature to be maintained at a constant level.

If a device malfunction prevents the control unit 200 from cooling the patient's skin by adjusting the treatment shot count, treatment irradiation time, or voltage applied to the thermoelectric element 330, the control unit 200 can emit an audible alarm and display a warning message via the output unit 240 to prompt the user to move the handpiece 130 away from the patient's skin. Additionally, the control unit 200 can display on the output unit 240 the time remaining until the handpiece is sufficiently cooled to provide the treatment shot count and treatment irradiation time.

The control unit 200 can acquire the treatment objective based on user input. The treatment objective may include at least one piece of information regarding the treatment area and procedure type. The control unit 200 can automatically provide at least one of the number of ultrasound treatment shots and the treatment irradiation time corresponding to the treatment objective.

For example, the treatment shot count and the treatment irradiation time may vary depending on the treatment area. The treatment area may include the skin of at least one of the patient's face, arms, legs, and abdomen. Skin thickness may vary depending on the treatment area. The control unit 200 may acquire pre-stored skin thickness values corresponding to the treatment area. The control unit 200 may acquire at least one of pre-stored treatment shot counts and treatment irradiation times corresponding to the skin thickness values.

According to various embodiments of the present disclosure, the skin treatment device 100 may include a sensor for measuring skin thickness. The control unit 200 may use the skin thickness value measured by the sensor to control the treatment shot count and the treatment irradiation time.

The skin treatment device 100 may measure skin thickness using a sensor that measures skin thickness. The thicker the skin measured by the sensor, the greater the treatment shot count and the longer the treatment irradiation time may be. The thinner the measured skin thickness, the fewer the treatment shot count and the shorter the treatment irradiation time.

Furthermore, the treatment shot count and the treatment irradiation time may vary depending on the type of procedure. The type of procedure may include information about the skin layer where the focus is generated, based on the purpose of the procedure, such as wrinkle treatment or fat removal. The deeper the skin layer where the focus is generated, the greater the treatment shot count and the longer the treatment irradiation time. The shallower the skin layer, the fewer the treatment shot count and the shorter the treatment irradiation time may be.

At least one of the first table, first function, second table, second function, third table, and third function may differ for at least one of the transducer frequency, the number of foci formed, and the treatment objective. Additionally, at least one of the first function, second function, and third function may differ for at least one of the transducer frequency, the number of foci formed, and the treatment objective. For example, the higher the transducer frequency, the greater the number of foci formed, and the deeper the skin layer corresponding to the treatment objective, the faster the skin temperature may increase. The higher the skin temperature, the fewer treatment shots and the shorter the treatment irradiation time the handpiece 130 can provide, and the voltage applied to the thermoelectric element 330 may increase. Therefore, at least one of the treatment shot count and the treatment irradiation time obtained through at least one of the first table, the first function, the second table, and the second function may decrease as the frequency of the transducer increases, as the number of foci formed increases, and as the depth of the skin layer corresponding to the treatment objective increases. The voltage applied to the thermoelectric element 330 obtained through the third table may increase as the frequency of the transducer increases, as the number of formed foci increases, and as the depth of the skin layer corresponding to the treatment objective increases.

The above describes the cooling of the handpiece of a skin treatment device using ultrasound, but is not limited thereto. For cooling the handpiece of a laser-based skin treatment device 100, the device may utilize at least one of functions or tables. The laser-based skin treatment device 100 may discharge cooling gas in addition to utilizing thermoelectric elements. Since the process of cooling the contact surface between the handpiece and the patient's skin using thermoelectric elements has already been described, redundant explanations are omitted.

The skin treatment device 100 using ultrasound or laser can discharge cooling gas to lower skin temperature when the contact surface between the patient's skin and the handpiece exceeds a predetermined temperature. In this regard, since the skin treatment device 100 of the present disclosure does not include a temperature sensor, it is necessary to determine the timing for discharging the cooling gas.

Typically, the skin treatment device 100 can determine the timing for discharging the cooling gas using at least one of a first table, a first function, a second table, and a second function.

More specifically, the control unit 200 can predict the temperature of the contact surface between the patient's skin and the handpiece (skin temperature). As the cumulative shot count and cumulative irradiation time of the handpiece increase, the skin temperature may rise. The control unit 200 can determine the skin temperature based on at least one of the cumulative shot count and cumulative irradiation time, using at least one of the first table, first function, second table, and second function. The control unit 200 can determine the skin temperature (V0) by applying the cumulative shot count obtained in step 910 of FIG. 9 and the cumulative irradiation time obtained in step 920 of FIG. 9 to at least one of the first table, first function, second table, and second function. Furthermore, the control unit 200 can discharge a predetermined amount of cooling gas when the skin temperature is equal to or above a predetermined threshold temperature. Additionally, the control unit 200 can determine the temperature after the cooling gas discharge based on the skin temperature and the predetermined amount of cooling gas. The temperature after the cooling gas is discharged becomes lower relative to the skin temperature as the predetermined amount of cooling gas increases. Furthermore, the temperature becomes significantly lower relative to the skin temperature as the difference between the current skin temperature and the predetermined temperature of the cooling gas increases.

For example, the temperature after discharge can be determined by the following equation:

$$Post - discharge temperature = ( k * skin temperature + m * cooling gas quantity * cooling gas temperature ) \text{} / ( k + m * cooling gas quantity )$$

Here, k and m may be predetermined real numbers. k is a predetermined value related to the heat capacity of the skin or handpiece, and m may be a predetermined value related to the specific heat of the cooling gas.

The control unit 200 can determine the skin temperature (V1) again by setting the post-discharge temperature to at least one T0 from the first table, first function, second table, and second function.

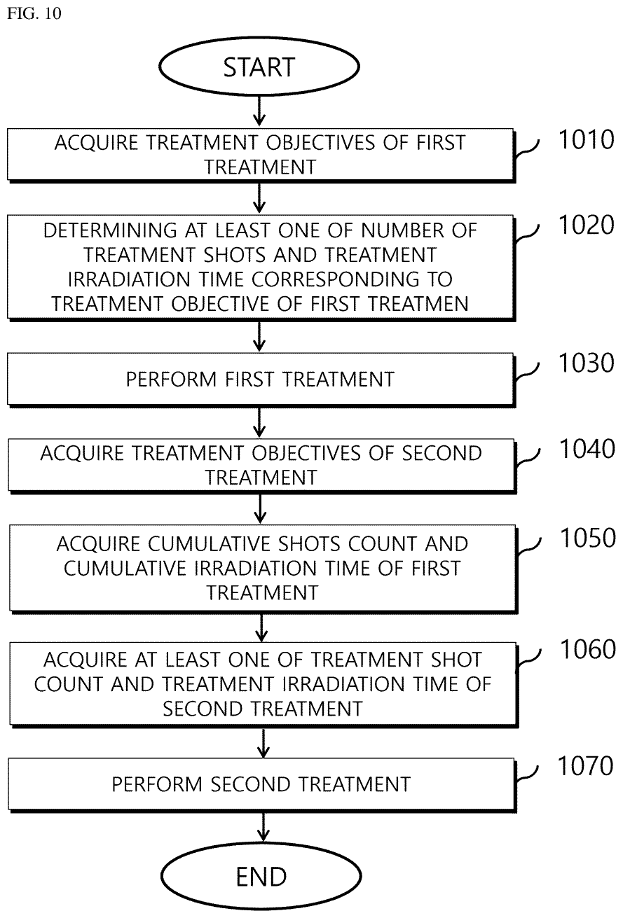

FIG. 10 is a flowchart illustrating the operation of a skin treatment device according to an embodiment of the present disclosure.

According to an embodiment of the present disclosure, when the skin treatment device 100 is powered on, it can begin the first treatment. The control unit 200 can perform a step 1010 of acquiring the treatment objective of the first treatment. The control unit 200 may perform a step 1020 of determining at least one of the treatment shot count and the treatment irradiation time corresponding to the treatment objective of the first treatment. The control unit 200 may automatically set the initial values for the treatment shot count and the treatment irradiation time based on the treatment objective. Additionally, the initial values for the treatment shot count and the treatment irradiation time may utilize values input by the user. The skin treatment device 100 may perform a step 1030 of performing the first treatment. The plurality of transducers 510, 520 may emit vibrational energy based on the set treatment shot count and the set treatment irradiation time. The thermoelectric element 330 can be energized based on the set treatment shot count and treatment irradiation time to cool the cooling surface. After completing all treatments according to the set treatment shot count and treatment irradiation time, or if the first treatment is forcibly terminated by the user, the control unit 200 can perform a second treatment. The second treatment may be a separate procedure that includes the same treatment objectives as the first treatment or different treatment objectives. The control unit 200 may perform a step 1040 of acquiring the treatment objectives for the second treatment. The control unit 200 may receive input for the treatment objectives of the second treatment. The control unit 200 may perform a step 1050 of acquiring at least one of the cumulative shot count and cumulative irradiation time of the first treatment. If the treatment objective of the second treatment is input, the time from the end of the first treatment to the time the treatment objective of the second treatment is input may be acquired as the procedure suspension time. The control unit 200 may perform the steps of FIGS. 7 to 9 using at least one of the cumulative shot count, cumulative irradiation time, and procedure suspension time of the first treatment. The control unit 200 may perform a step 1060 of acquiring at least one of the treatment shot count and treatment irradiation time for the second treatment. The control unit 200 may determine at least one of the treatment shot count and the treatment irradiation time for the second treatment. The control unit 200 may determine the voltage applied to the thermoelectric element 330 for at least one of the treatment shot count and the treatment irradiation time of the second treatment. The control unit 200 may perform a step 1070 of performing the second treatment. The plurality of transducers 510, 520 may emit vibrational energy based on the determined treatment shot count and treatment irradiation time. The thermoelectric element 330 may cool the cooling surface based on the determined voltage.

Due to errors in the treatment shot count and treatment irradiation time, and individual patient skin differences, the patient may feel heat or pain during the procedure. The control unit 200 may determine that treatment has been temporarily interrupted if the handpiece 130 is lifted away from the patient's skin. The control unit 200 may automatically readjust the treatment shot count and treatment irradiation time or receive input of the treatment shot count and treatment irradiation time from the user. The control unit 200 can acquire the cumulative shot count and cumulative irradiation time based on the changed treatment shot count and treatment irradiation time. The newly acquired cumulative shot count and cumulative irradiation time can be applied to the treatment shot count and treatment irradiation time for subsequent treatments.

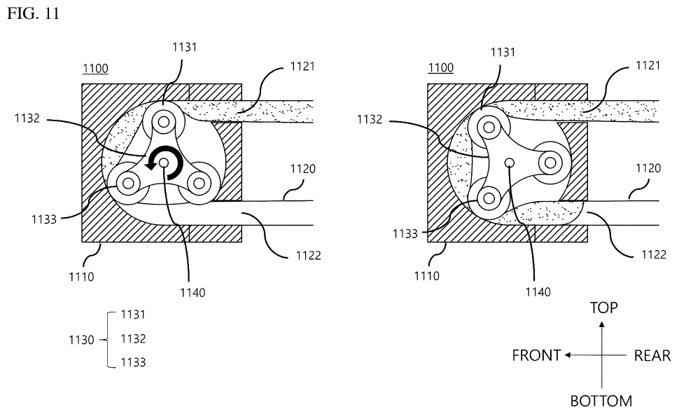

FIG. 11 is a diagram illustrating a drug supply unit according to an embodiment of the present disclosure.

The drug supply unit 1100 may include a pressurized housing 1110 having an inlet 1121 coupled to the drug storage unit and an outlet 1122 coupled to the drug injection unit. The inlet 1121 and outlet 1122 may be formed on the same surface on one side of the pressurized housing 1110.

The drug supply unit 1100 may be located inside the pressurized housing 1110 and may include a rubber tube 1120 coupling the inlet 1121 and outlet 1122 and transferring the drug.

The drug supply unit 1100 may be located inside the pressurized housing 1110. The drug supply unit 1100 may be rotatably coupled to the pressurized housing 1110 about the drive shaft 1140. The drug supply unit 1100 may include a rotating unit 1130 comprising a plurality of rollers 1131, a rotary plate 1132, and a roller support unit 1133. The rotating unit 1130 may include a rotary plate 1132, a roller support unit 1133, and a plurality of rollers 1131. The rotary plate 1132 may have protrusions formed at one of the intervals of 72 degrees, 90 degrees, 120 degrees, and 180 degrees around the drive shaft 1140. However, this is not limited thereto, and the angle between the protrusions may be any predetermined arbitrary angle. The roller support unit 1133 may be coupled to a protrusion of the rotary plate 1132 and may be coupled to a rotation shaft 1134 of one of the plurality of rollers 1131. One of the plurality of rollers 1131 may be coupled to the roller support unit 1133. One roller 1131 may be coupled one-to-one to one roller support unit 1133.

The drug supply unit 1100 may include a drive shaft 1140 penetrating the center of the rotating unit 1130 and a drive unit that rotates the rotating unit 1130 about the drive shaft 1140. The drive unit may include a motor. The drive shaft 1140 may be coupled to the drive unit.

At least one surface inside the pressurized housing 1110 may include a curved surface. More specifically, the surface opposite the surface where the inlet 1121 and outlet 1122 are formed may be a curved surface having an arc. At least one roller among the plurality of rollers included in the rotating unit may be in contact with the curved surface inside the pressurized housing 1110 to compress the rubber tube. When the rotating unit rotates, the rollers may rotate along the curved surface inside the pressurized housing.

A space may be formed inside the rubber tube 1120 between the pressurized housing 1110 and the plurality of rollers 1131 as the pressurized housing 1110 and the plurality of rollers 1131 compress the rubber tube 1120. This space may contain a drug. Since the drug supply unit 1100 rotates while the pressurized housing 1110 and the plurality of rollers 1131 compress the rubber tube 1120 that transports the drug as the rotation unit 1130 rotates, the drug inside the rubber tube 1120 may also rotate while being separated. The separated drug can be delivered to the patient's skin through the rubber tube 1120. The amount of drug to be injected may be predetermined for each type of procedure performed by the skin treatment device 100. The control unit 200 may store the rotational angle of the drug supply unit 1100 corresponding to the predetermined amount of drug. The larger the rotation angle of the drug supply unit 1100, the larger the predetermined amount of drug can be. The drug supply unit 1100 can supply a predetermined amount of drug to the patient by rotating the rotating unit 1130 to the predetermined rotation angle. The drug supply unit 1100 can accurately deliver different amounts of drug to the patient's skin for each type of ultrasound procedure.

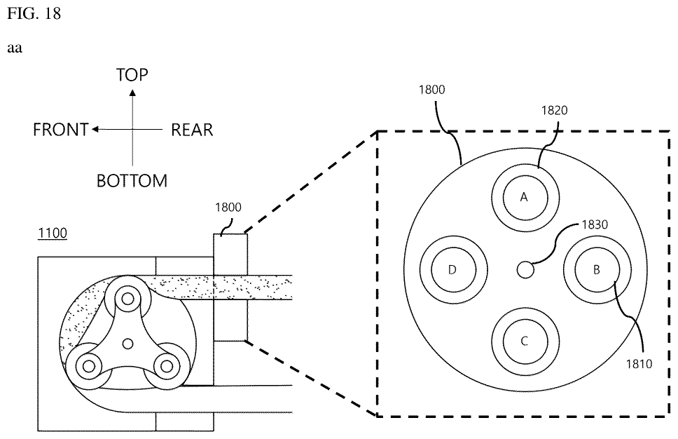

FIG. 18 is a diagram showing a drug supply unit according to an embodiment of the present disclosure.

According to various embodiments of the present disclosure, the drug storage unit can store a plurality of different drugs. The plurality of drugs may each have a separate rubber tube 1120. Referring to FIG. 18, the drug supply unit 1100 may include a circular rubber tube fixing unit 1800. The rubber tube fixing unit 1800 may fix a plurality of rubber tubes 1120. Within the rubber tube fixing unit 1800, the plurality of rubber tubes 1120 may be arranged in a circular pattern along a fixing unit rotation shaft 1830.

The rubber tube fixing unit 1800 may include a plurality of fixing units 1810 that fix rubber tubes 1120 separately according to the drug. For example, if drugs A, B, C, and D are stored in the drug storage unit, the rubber tube fixing unit 1800 may include fixing units 1810 corresponding to A, B, C, and D, respectively. Thus, the rubber tubes coupled to A, B, C, and D can be fixed to the A, B, C, and D fixing units 1810 of the rubber tube fixing unit 1800, respectively.

With respect to the inlet of the drug supply unit 1100, the rubber tubes coupled to the drug storage unit and the drug supply unit 1100 and the rubber tubes coupled to the drug supply unit 1100 and the drug injection unit 410 may be separated or connected. The rubber tube fixing unit 1800 may include a plurality of coupling units 1820 to which a plurality of rubber tubes 1120 can be coupled to the inlet of the drug storage unit. The rubber tube fixing unit 1800 may include a fixing unit rotation shaft 1830. The rubber tube fixing unit 1800 can rotate relative to the drug supply unit 1100 about the fixing unit rotation shaft 1830. The inlet 1121 of the drug supply unit 1100 can be coupled to one of a plurality of different rubber tubes 1120 via the coupling unit 1820 of the rubber tube fixing unit 1800.

The skin treatment device 100 can receive input of a plurality of drug information stored in the drug storage unit from the user via the input unit 250. The control unit 200 may store different drug information for each type of procedure the skin treatment device 100 can perform.

When the user initiates a treatment, the control unit 200 can acquire the drug information corresponding to the treatment type. The control unit 200 can then start supplying the drug corresponding to that drug information from the drug storage unit. The control unit 200 can rotate the rubber tube fixing unit 1800 to couple the rubber tube corresponding to the drug information to the drug supply unit 1100. Therefore, the drug supply unit 1100 can appropriately provide drugs suitable for various treatment types.

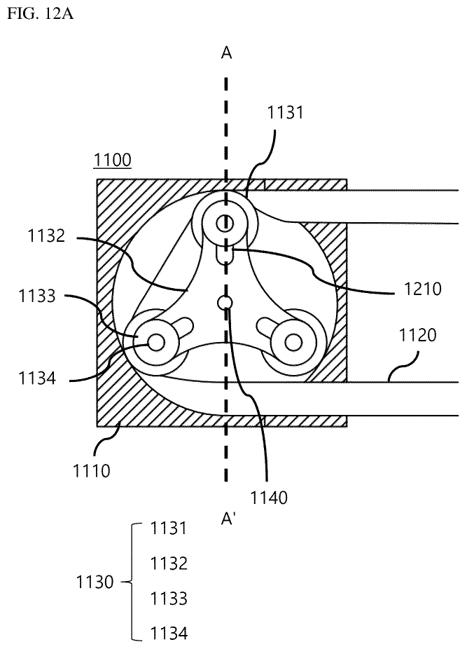

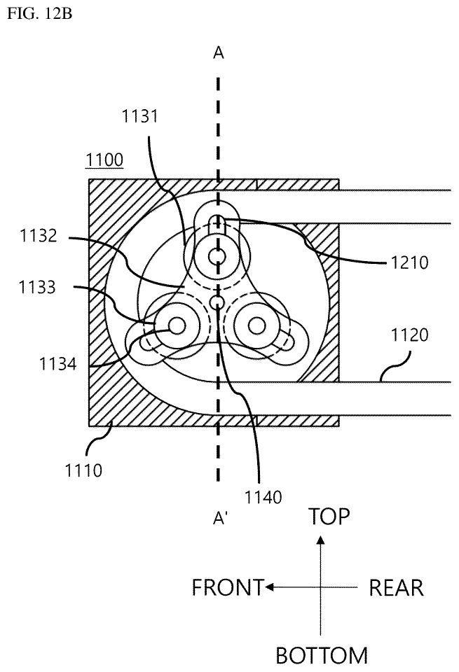

FIGS. 12A and 12B are diagrams illustrating the operation of the drug supply unit according to an embodiment of the present disclosure.

When the medical skin treatment device is deactivated, the drug supply unit 1100 can retract a plurality of rollers 1131 into the interior of the rotary plate 1132, so as not to apply pressure to the rubber tube 1120. The control unit 200 can determine the deactivation of the skin treatment device 100. The control unit 200 can determine that the skin treatment device 100 is deactivated when it receives input from the user to turn off the power to the skin treatment device 100. Furthermore, the control unit 200 can determine that the skin treatment device 100 is deactivated if the skin treatment device 100 does not operate for a predetermined period of time.

When the medical skin treatment device is activated, a plurality of rollers 1131 protrude outward from the rotary plate 1132, allowing the pressurized housing 1110 and rollers 1131 to apply pressure to the rubber tube 1120. The control unit 200 can determine the activation of the skin treatment device 100. When the control unit 200 receives a new operation input from the user while the skin treatment device 100 is in a deactivated state, it can determine that the skin treatment device 100 has been reactivated.

A plurality of moving holes 1210 may be formed in the plurality of protrusions of the rotary plate 1132. A single roller 1131, roller support unit 1133, and rotation shaft 1134 may be coupled to a single moving hole 1210.

The following describes the structure whereby the rollers protrude from or are retracted into the rotary plate.

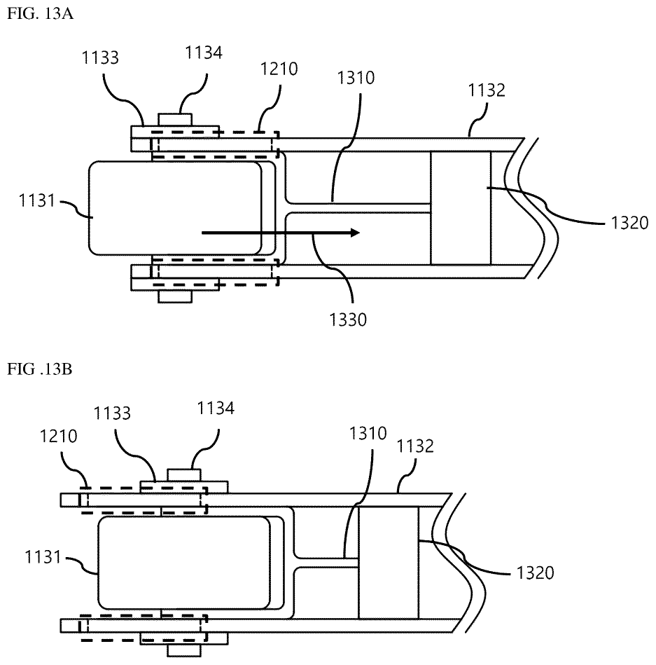

FIGS. 13A and 13B are diagrams illustrating the operation of the rotating unit according to an embodiment of the present disclosure.

FIGS. 13A and 13B may represent the A-A′ section of FIGS. 12A and 12B. Referring to FIGS. 12A, 12B, 13A, and 13B, the rotating unit 1130 may include a connecting rod 1310 and a storage drive unit 1320. The connecting rod 1310 may be coupled to the rotation shaft 1134 and the storage drive unit 1320. Driven by the storage drive unit 1320, the rotation shaft 1134 can move along the moving hole 1210 in a first direction 1330 and in the opposite direction of the first direction 1330. The first direction 1330 may refer to the direction toward the center of the rotary plate 1132, the direction in which the roller 1131 is stored, or the direction toward the drive shaft 1140. The opposite direction of the first direction 1330 may refer to the direction in which the roller 1131 protrudes outside the rotary plate 1132.

Referring to FIG. 13A, the roller 1131 may be positioned outside the rotary plate 1132. At this time, the roller 1131 may be in a state where it is pressed against the rubber tube 1120 while being in close contact with the pressurized housing 1110. When the skin treatment device 100 is deactivated, the control unit 200 can control the storage drive unit 1320. The storage drive unit 1320 may include a motor. The storage drive unit 1320 may include a rack coupled to the motor and a pinion coupled to the connecting rod 1310. Through the operation of the motor in the storage drive unit 1320, the connecting rod 1310 can perform linear reciprocating motion. Therefore, the rotation shaft 1134 can move in the first direction 1330 along the moving hole 1210 by the storage drive unit 1320. The length of the moving hole 1210 may be equal to or greater than the diameter of the rubber tube 1120.

Referring to FIG. 13B, the rotation shaft 1134 may move in the first direction 1330 along the moving hole 1210, causing the roller 1131 to be housed inside the rotary plate 1132. At this time, the roller 1131 may not be in close contact with the pressurized housing 1110 or, at least, may not apply pressure to the rubber tube 1120. By not pressing or stretching the rubber tube 1120 when the skin treatment device is in an inactive state, the service life of the rubber tube can be extended.

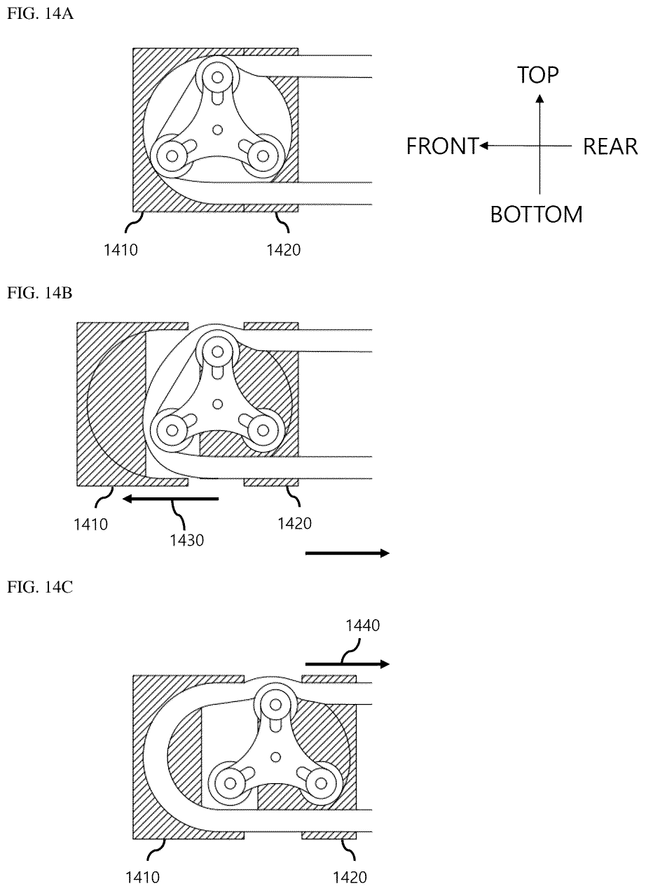

FIGS. 14A, 14B, and 14C are diagrams illustrating the operation of the drug supply unit according to an embodiment of the present disclosure.

Referring to FIG. 14A, the drug supply unit 1100 may be in a state where the pressurized housing 1110 and roller 1131 are compressing the rubber tube. The pressurized housing 1110 of the drug supply unit 1100 may include a pressurized housing moving unit 1410 and a pressurized housing base unit 1420. At least one of the main body 110 of the skin treatment device 100 and the pressurized housing 1110 may include a housing drive unit. The housing drive unit may be coupled to at least one of the pressurized housing moving unit 1410 and the pressurized housing base unit 1420. The housing drive unit can move the pressurized housing moving unit 1410 and the pressurized housing base unit 1420 in the front-rear direction.

Referring to FIG. 14B, when the medical skin treatment device 100 is deactivated, the drug supply unit 1100 may move the pressurized housing moving unit 1410 included in the pressurized housing 1110 forward 1430 with respect to the pressurizing housing base unit 1420 included in the pressurized housing 1110 by the housing drive unit so as not to apply pressure to the rubber tube. That is, the pressurized housing 1110 may move away from the roller included in the rotating unit 1130 so as not to apply pressure to the rubber tube.

When the medical skin treatment device 100 is activated, the housing drive unit can cause the pressurized housing moving unit 1410 to retract 1440 relative to the pressurizing housing base unit 1420, to apply pressure to the rubber tube. At this time, the pressurized housing moving unit 1410 can be re-coupled to the pressurized housing base unit 1420 as shown in FIG. 14A.

Referring to FIG. 14C, the drug supply unit 1100 may not apply pressure to the rubber tube when the medical skin treatment device 100 is deactivated, as the rotating unit 1130 retracts 1440. The pressurized housing base unit 1420 may be coupled to the rotating unit 1130 via the drive shaft 1140 of the rotating unit 1130. Retraction 1440 of the pressurized housing base unit 1420 by the housing drive unit allows the roller included in the rotating unit 1130 to move away from the rubber tube, thereby not applying pressure.

When the medical skin treatment device 100 is activated, the rotating unit 1130 may move forward 1430 to apply pressure to the rubber tube. The pressurized housing base unit 1420 can be move forward 1430 again by the housing drive unit, allowing the pressurized housing base unit to be coupled to the pressurized housing moving unit 1410 as shown in FIG. 14A. Consequently, the rollers contained within the rotating unit 1130 can again apply pressure to the rubber tube. 1130.

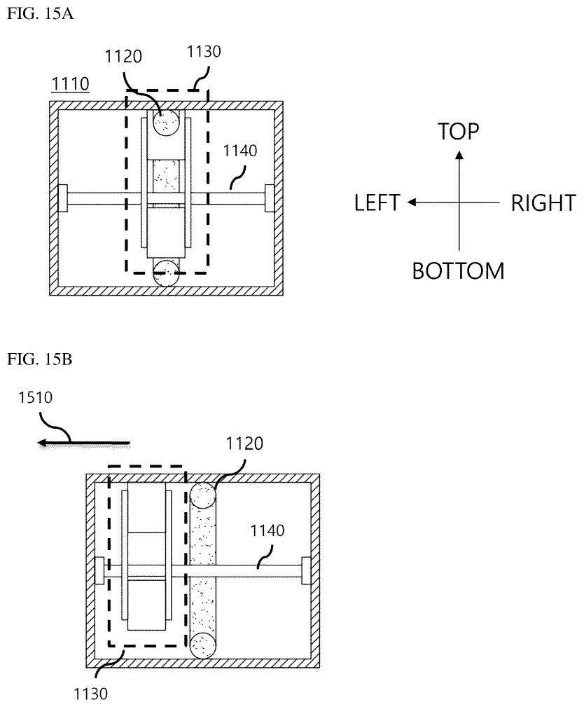

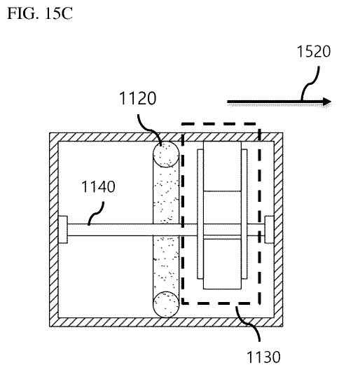

FIGS. 15A, 15B, and 15C are diagrams illustrating the operation of the drug supply unit according to an embodiment of the present disclosure.

Referring to FIG. 15A, the drug supply unit 1100 may be in a state where the pressurized housing 1110 and roller 1131 are pressing against the rubber tube 1120. The pressurized housing 1110 may include a space extended sufficiently to the left and right to accommodate the rotating unit. The rotating unit 1130 drive shaft 1140 may pass through the interior space of the pressurized housing 1110. At least one of the main body 110 and the rotating unit 1130 of the skin treatment device 100 may include a rotating part drive unit. The rotating part drive unit may move the rotating unit 1130 laterally along the rotating part drive shaft 1140.

Referring to FIG. 15B, when the medical skin treatment device 100 is deactivated, the drug supply unit 1100 may not apply pressure to the rubber tube as the rotating unit moves in the left direction 1510. When the medical skin treatment device 100 is activated, the rotating unit 1130 can move in the right direction 1520 to apply pressure to the rubber tube again, as shown in FIG. 15A.

Referring to FIG. 15C, the drug supply unit 1100 may not apply pressure to the rubber tube when the medical skin treatment device 100 is deactivated, as the rotating unit moves in the right direction 1520. When the medical skin treatment device 100 is activated, the rotating unit 1130 can move in the left direction 1510 to reapply pressure to the rubber tube as shown in FIG. 15A.

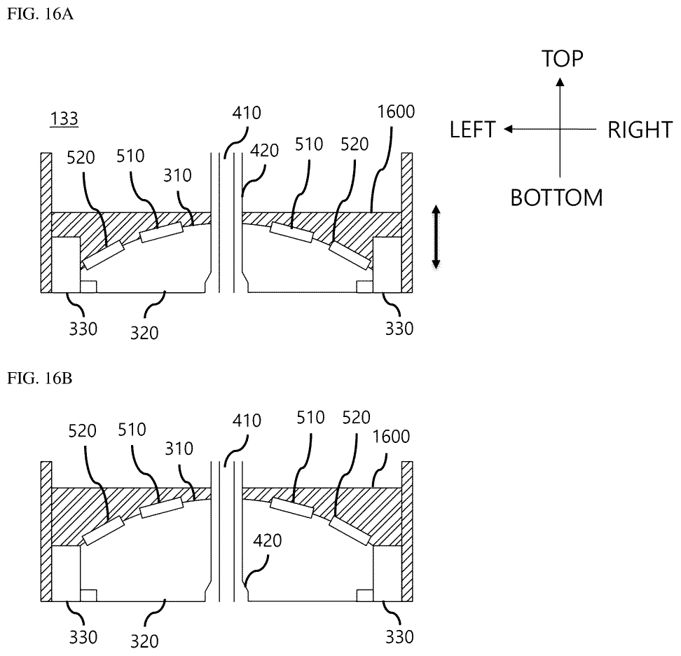

FIGS. 16A and 16B are diagrams showing a base unit according to an embodiment of the present disclosure.

The skin treatment device 100 can move the transducer placement surface 310 up and down based on the depth of focus input by the user, thereby changing the depth of focus of the transducers 510, 520. The base unit 133 may include an placement surface drive unit 1600. The placement surface drive unit 1600 may include a motor. The placement surface drive unit 1600 may include a rack coupled to the motor and a pinion formed on the transducer placement surface 310. Through the operation of the motor in the placement surface drive unit 1600, the transducer placement surface 310 can perform a vertical reciprocating motion. Therefore, the placement surface drive unit 1600 can move the transducer placement surface 310 vertically along the wall surface of the base unit 133 and the skin suction unit 420.

The control unit 200 of the skin treatment device 100 can acquire the depth of focus for at least one of the plurality of transducers 510, 520 based on user input. The control unit 200 may store depth values corresponding to treatment objectives. If the user does not input the depth of focus, the control unit 200 may automatically acquire the depth of the focus corresponding to the treatment objective. The control unit 200 may store the range of the focus corresponding to the position of the transducer placement surface 310.

If the depth of the focus falls outside the range of focus corresponding to the current position of the transducer placement surface 310, the transducer placement surface 310 can move vertically to shift the focus of the plurality of transducers 510, 520.

If the user-set focus depth is smaller than the focus range corresponding to the current position of the transducer placement surface 310, the transducer placement surface 310 can move upward. By moving the transducer placement surface 310 upward, the plurality of transducers 510, 520 move together, reducing the depth of the focus formed by the plurality of transducers 510, 520, enabling treatment of shallower areas.

If the user-set depth of focus is greater than the focal range corresponding to the current position of the transducer placement surface 310, the transducer placement surface 310 can move downward. By moving the transducer placement surface 310 downward, the plurality of transducers 510, 520 move together, increasing the depth of the focus formed by the plurality of transducers 510, 520, enabling treatment of deeper areas.

If the user-set depth of focus falls outside the range of depth of focuss reachable by the movement of the transducer placement surface 310, the output unit 240 can be controlled to emit an audible warning and a warning message prompting the user to reset the depth of focus.

Thus far, various embodiments have been described. Those skilled in the art to which the present invention pertains will understand that the invention may be implemented in modified forms without departing from its essential character. Therefore, the disclosed embodiments should be considered from an illustrative perspective, not a limiting one. The scope of the invention is defined not by the foregoing description but by the appended claims, and all differences within the scope of the claims should be interpreted as falling within the scope of the invention.

Meanwhile, the above-described embodiments of the present invention can be written as programs executable on a computer and implemented on a general-purpose digital computer operating said program using a computer-readable recording medium. The computer-readable storage medium includes storage media such as magnetic storage media (e.g., ROM, floppy disk, hard disk, etc.) and optical read media (e.g., CD-ROM, DVD, etc.).

What is claimed is:

1. A medical skin treatment device, comprising:
a main body including a control unit for controlling an operation of the skin treatment device, a power supply unit for supplying power to the medical skin treatment device, and a drug storage unit for storing a drug to be injected into a patient's skin;
a transport unit for moving the main body;
a handpiece supplied with power from the power supply unit of the main body, controlled by the control unit, and injecting the drug into the patient's skin to improve the skin; and
a drug supply unit coupled to the drug storage unit and supplying a predetermined amount of drug to the handpiece,
wherein the handpiece comprises:
a base unit coupled to one side of the handpiece and including a transducer placement surface;
a drug injection unit coupled to the drug supply unit, positioned at the center of the base unit, and injecting the drug into the patient's skin;
a skin suction unit formed along an outer periphery of the drug injection unit, which sucks in the patient's skin;
a filter for filtering drugs and waste materials sucked in by the skin suction unit; and
a ring-shaped transducer formed along the outer periphery of the skin suction unit, which vibrates at a predetermined treatment frequency to deliver vibrational energy to the patient's skin.

2. The medical skin treatment device of claim 1,
wherein the drug supply unit comprises:
a pressurized housing having an inlet coupled to the drug storage unit and an outlet coupled to the drug injection unit;
a rubber tube located inside the pressurized housing, coupling the inlet and outlet and conveying the drug;
a rotating unit positioned inside the pressurized housing, rotatably coupled to the pressurized housing about a drive shaft, and including a plurality of rollers, a rotary plate, and a roller support unit; and
a drive unit including the drive shaft penetrating the center of the rotating unit, which rotates the rotating unit about the drive shaft;
wherein, as the rotating unit rotates, the pressurized housing and the plurality of rollers compress at least a portion of the rubber tube conveying the drug, thereby separating a predetermined amount of drug between the plurality of rollers; and
wherein the rotating unit comprises a rotary plate having a protrusion formed thereon, a roller support unit coupled to the protrusion of the rotary plate and coupled to a rotation shaft of one of the plurality of rollers, and one of the plurality of rollers coupled to the roller support unit.

3. The medical skin treatment device of claim 2,
wherein the drug supply unit is configured to:
when the medical skin treatment device is deactivated, retract the plurality of rollers into an interior of the rotary plate so as not to apply pressure to the rubber tube, and
when the medical skin treatment device is activated, protrude the plurality of rollers outward from the rotary plate to apply pressure to the rubber tube.

4. The medical skin treatment device of claim 2,
wherein the drug supply unit is configured to:
when the medical skin treatment device is deactivated, retract the rotating unit so as not to apply pressure to the rubber tube, and
when the medical skin treatment device is activated, move forward the rotating unit to apply pressure to the rubber tube.

5. The medical skin treatment device of claim 2,
wherein, when the medical skin treatment device is deactivated, a pressurized housing moving unit included in the pressurized housing moves forward relative to the pressurized housing base unit included in the pressurized housing by a housing drive unit, so as not to apply pressure to the rubber tube, and
when the medical skin treatment device is activated, the housing drive unit causes the pressurized housing moving unit to retract relative to the pressurized housing base unit to apply pressure to the rubber tube,
wherein the housing drive unit is coupled to the pressurized housing base unit.

6. The medical skin treatment device of claim 1,
wherein the skin suction unit is configured to:
suck in the patient's skin to form negative pressure,
include a pressure sensor for measuring the pressure formed inside the skin suction unit,
wherein the control unit is configured to:
control the drug injection unit to stop injecting the drug when the pressure formed inside the skin suction unit is equal to or greater than a critical pressure, and
control the drug injection unit to inject the drug when the pressure in the skin suction unit is less than the critical pressure.

7. The medical skin treatment device of claim 6,
wherein the control unit is configured to:
control the transducer not to emit vibrational energy when the pressure formed inside the skin suction unit is equal to or greater than the critical pressure, and
control the transducer to emit vibrational energy when the pressure in the skin suction unit is less than the critical pressure.

8. The medical skin treatment device of claim 1,
wherein the transducer placement surface moves up and down based on a depth of focus input by a user, thereby changing the depth of focus of the transducer.
